(19) United States
(12) Patent Application Publication
(10) Pub. No.: US 2015/0335752 A1
(43) Pub. Date: Nov. 26, 2015

(54) Temperature Stable Vaccine Formulations

(71) Applicant: Emergent Product Development Gaithersburg Inc., Gaithersburg, MD (US)

(72) Inventors: Jee Look, Boyds, MD (US); Christian Fernando Ruiz, Germantown, MD (US); Aaron Paul Miles, Potomac, MD (US); Richard William Welch, Gaithersburg, MD (US)

(73) Assignee: Emergent Product Development Gaithersburg Inc., Gaithersburg, MD (US)

(21) Appl. No.: 14/410,942

(22) PCT Filed: Jun. 25, 2013
(86) PCT No.: PCT/US13/47712

§ 371 Date: Dec. 23, 2014

Related U.S. Application Data
(60) Provisional application No. 61/664,062, filed on Jun. 25, 2012.
(60) Provisional application No. 61/801,385, filed on Mar. 15, 2013.

(51) Int. Cl. A61K 47/26; A61K 39/07; A61K 9/19; A61K 47/02

(52) U.S. Cl. CPC A61K 47/26; A61K 47/02; A61K 39/07; A61K 9/19; A61K 2039/55505

(57) ABSTRACT

Formulations of vaccine antigen, such as anthrax protective antigen, are provided that are stable after undergoing freeze and thaw conditions. Methods of using the formulations to prepare vaccine are also provided. Vaccines comprising the formulations are useful, for example, to protect against, inhibit or alleviate a disease or infection, such as related to anthrax infection.

### GOVERNMENT RIGHTS

This invention was made in part with government support under grant HHSO100201000059C. The government may have certain rights in this invention.

### REFERENCE TO SEQUENCE LISTING SUBMITTED ELECTRONICALLY

The content of the electronically submitted sequence listing in ASCII text file (Name “2479115PC02_sequencelisting.txt”; Size: 12,954 bytes; and Date of Creation: Jun. 25, 2013) filed with the application is incorporated herein by reference in its entirety.

### DESCRIPTION OF THE INVENTION

1. Field of the Invention

The present invention relates to temperature stable vaccine formulations containing an antigen adsorbed to an aluminum adjuvant and methods of preparing such formulations. The invention includes lyophilized and frozen vaccine formulations. The invention includes temperature stable vaccines, methods of making temperature stable vaccines and methods of use.

2. Background of the Invention

Anthrax is a well-known infectious disease caused by a Gram-positive bacterium, Bacillus anthracis (B. anthracis). Among the three types of anthrax infection (cutaneous, gastrointestinal, and inhalation), cutaneous anthrax is the most common and is relatively easily treatable with various antibiotics. The other two types of anthrax infections are rare, but usually fatal even with aggressive anti-microbial therapy.

The major virulence factor, anthrax toxin, is composed of three proteins: protective antigen (PA, 83 kilo Dalton, kDa), edema factor (EF, 89 kDa), and lethal factor (LF, 90 kDa). The toxin components act in the binary combinations of PA+EF (edema toxin), and PA+LF (lethal toxin). PA is a cell receptor-binding protein and delivers the other two proteins (EF and LF) into the cytosol of infected cells.

The most effective known method for preventing anthrax is vaccination. The current and only FDA-approved anthrax vaccine in the United States (produced by Emergent BioSolutions Inc. under the trademark BioThrax® (Anthrax Vaccine Adsorbed)) is produced from a sterile cell-free filtrate from an avirulent B. anthracis V770-NP1-R strain. The licensed anthrax vaccine is also called Anthrax Vaccine Adsorbed (or AVA). The vaccine primarily consists of PA, and aluminum hydroxide is used as an adjuvant. The vaccine was developed during the 1950s and 1960s and is licensed by the FDA to Emergent BioSolutions Inc. The vaccine shows less than 0.06% systemic reactions. The ability of the vaccine to elicit an immune response in humans is well-documented. The AVA vaccine is currently licensed for five doses over 18 months followed by annual boosts.

Although the AVA vaccine is effective and safe, new immunogenic compositions for preparing a vaccine that protects a subject against a lethal B. anthracis infection using recombinant technologies are under development. Because protective antigen (PA) is the common factor required for both the actions of LF and EF, it is often used to prepare vaccines for anthrax. Examples of PA vaccines in development include those disclosed in U.S. Pat. Nos. 6,316,006 and 6,387,665 and patent applications US 2010/0183675, US2011/0229507 and WO2010/053610.

Vaccines such as an AVA and PA typically contain at least one adjuvant to enhance a subject's immune response. Aluminum salt adjuvants, frequently referred to as alum, are currently the most widely used adjuvant for use in humans. Alum is usually aluminum hydroxide (also marketed as ALHYDROGEL® (aluminum hydroxide) or aluminum phosphate). AVA and the “next generation” Anthrax vaccines (such as recombinant PA) are formulated with ALHYDROGEL which binds the antigen.

Currently, vaccines containing alum require a cold chain. Cold chains have been established globally to keep vaccines at 2-8° C. during storage and distribution. Maintaining cold chains is expensive and difficult. In the event of a cold chain failure, vaccines can be exposed to higher or lower temperatures than intended. It is generally recommended that vaccines that contain alum be discarded if they undergo freeze/thaw processing during shipping and storage. Failure of a cold chain can occur in both industrialized and developing nations, and there are many reasons for cold chain failure, for instance, equipment failure, lack of resources or poor compliance. In many developing countries such as Indonesia, freezing temperatures were recorded in 75% of baseline shipments and freezing of freeze-sensitive is widespread. See Hepatitis B vaccine freezing in the Indonesian cold chain: evidence and solutions. Bulletin of the World Health Organization 2004; 82:99-105.

A vaccine that is dependent on a cold chain may also take longer to distribute to those in need in a timely manner. In the event of a bioterrorist event or other public health emergency, the ability to rapidly deliver vaccines and other medical countermeasures is critical. Eliminating dependence on the cold chain for distribution would lead to more prompt and efficient delivery of medical countermeasures in a variety of climates.

In order to avoid or mineralize cold chain requirements, many licensed vaccines are formulated as a dry powder composition that can be reconstituted immediately prior to administration. To date, all dry powder vaccines licensed for use in the US are produced through a lyophilization process. Lyophilization, also referred to as freeze drying, is a process that improves the long term stability of a vaccine. The process involves freezing the liquid vaccine formulation and subliming the frozen formulation under vacuum. Other technologies such as spray drying and foam drying have been developed with the aim of producing a stable, dry powder vaccine. These newer technologies produce dry powder vaccine material without the need for freezing and can be used with an alum containing vaccine. See, for instance, Chen et al., 2010, Vaccine 28:5093-5099. However, these newer technologies are still in their infancy and have yet to be used in the production of a licensed vaccine in the United States.

Freezing of vaccine compositions containing alum (either as part of the lyophilization process or to produce a frozen vaccine) generally induces aggregation of the aluminum particles and causes degradation of the antigen adsorbed onto the alum adjuvant resulting in potency loss. In addition, freezing causes reduction of the height of the settled aluminum gel (commonly referred to as gel collapse). See, for instance, “The effect of freezing on the appearance, potency and toxicity of adsorbed and unadsorbed DPT vaccines,” 1980, WHO Weekly Epidemiological Record 55:385-92; “Temperature Sensitivity of Vaccines,” August 2006, WHO publication WHO/IVB/06.10; Diminsky et al., 1999, Vaccine 18(1-2):3-17; Maa et al., 2003, J Pharm Sci 92(2):319-332.

Accordingly, there is a need to produce a vaccine that contains alum that can withstand freezing. Such a vaccine may be subjected to freezing as part of the manufacturing process (e.g., a lyophilized or frozen vaccine), shipping process or during storage. The present invention discloses novel formulations for the production of temperature stable vaccines containing alum.

### SUMMARY OF THE INVENTION

The present invention provides vaccine formulations that contain alum and are capable of being frozen with little to no reduction of potency. In one embodiment, the frozen vaccine composition exhibits little to no alum gel collapse as a result of freezing.

In one embodiment, the vaccine or composition comprises at least 20% sugar which acts as a stabilizer. In one embodiment, the vaccine or composition comprises greater than 15%, greater than 20%, greater than 25%, or greater than 30% sugar. In some embodiments, the amount of sugar can be reduced without compromising potency if additional stabilizing agents such as amino acids and/or surfactants are added. For frozen and lyophilized vaccine formulations, potency can also be improved by increasing the freezing rate and by freezing suspended particles (as opposed to settled particles).

The invention includes frozen vaccine compositions, lyophilized vaccine compositions (which undergo freezing as part of the lyophilization process) and other vaccine formulations that are not susceptible to freeze/thaw conditions during shipping and storage.

Some embodiments of the invention include a composition for preparation of a lyophilized vaccine comprising at least one antigen adsorbed to an aluminum adjuvant and at least 20% (w/v) non-reducing sugar. Another embodiment includes a temperature stable liquid vaccine composition comprising at least one antigen adsorbed to an aluminum adjuvant and at least 20% (w/v) sugar. A further embodiment includes a composition comprising, prior to lyophilization, at least one antigen absorbed to an aluminum adjuvant and at least 20% (w/v) non-reducing sugar, wherein after reconstitution the non-reducing sugar is at least 6% (w/v). Compositions of the invention may further comprise a surfactant. In some embodiments composition of the invention comprises at least one antigen adsorbed to an aluminum adjuvant, a surfactant and at least 15% (w/v) sugar can be used for preparation of a lyophilized vaccine.

The invention also includes temperature stable liquid vaccine compositions comprising at least one antigen adsorbed to an aluminum adjuvant, a surfactant and at least 15% (w/v) sugar. In some embodiments, a composition further comprises an amino acid. Also included are stable liquid vaccine compositions comprising at least one antigen adsorbed to an aluminum adjuvant, a surfactant, an amino acid and at least 10% (w/v) sugar.

The invention further provides compositions for preparation of a lyophilized vaccine comprising at least one antigen adsorbed to an aluminum adjuvant, a surfactant, an amino acid and at least 10% (w/v) sugar.

The invention can be applied to numerous vaccines that contain an antigen adsorbed to an aluminum adjuvant. In one embodiment the vaccine is an anthrax vaccine such as rPA or Anthrax Vaccine Adsorbed.

The source of the protective antigen may vary. Thus, in some embodiments, the B. anthracis protective antigen protein is produced from an asporogenic B. anthracis bacterium. In some embodiments, the asporogenic B. anthracis bacterium is a ΔSterne-1(pPA102) CR4 strain of bacteria. In some embodiments, PA protein is expressed in other organisms such as E. coli.

In some embodiments, compositions of the invention may further comprise adjuvants (e.g., in addition to aluminum).

The present invention includes methods of preventing and treating an anthrax infection comprising administering to a subject a pharmaceutically effective amount of one of the vaccines of the invention. In another embodiment, the invention includes methods of inducing an immune response in a subject comprising administering to the subject a vaccine of the invention.

The present invention provides method for lyophilizing a vaccine comprising (i) freezing a composition of the invention and (ii) subjecting the frozen composition to sublimation.

### BRIEF DESCRIPTION OF THE DRAWINGS

FIG. 1. A photograph of an rPA vaccine without a sugar stabilizer at 2°-8° C. compared to the same composition frozen at −80° C. and thawed.

FIG. 2. A photograph of an rPA vaccine with 20% trehalose and 2% arginine at 2°-8° C. compared to the same composition frozen at −80° C. and thawed.

FIG. 3. Photographs of an rPA vaccine with and without sucrose at 2°-8° C. and at −80° C. followed by thawing.

FIG. 4. A graph showing the GeoMean NF50 of a no sugar rPA formulation before and after freezing and photographs showing the collapse of the alum gel.

FIG. 5. A graph showing the GeoMean NF50 response of lyophilized samples with and without sugar that were not frozen.

FIG. 6. A graph showing the GeoMean NF50 before and after freeze/thaw for an rPA composition containing 20% trehalose.

FIG. 7. A graph showing GeoMean NF50 for all lyophilized samples compared to a liquid control.

FIGS. 8A-B. A graph showing NF50 over time for lyophilized rPA at (A) 5° C. and 50° C. compared to AVA shown in linear NF50 scale and (B) 5° C. and 50° C. compared to AVA shown in log NF50 scale, each vaccine was at a 1:4 dilution.

FIGS. 9A-B. A graph showing NF50 over time for lyophilized rPA at (A) 5° C. and 50° C. compared to AVA shown in linear NF50 scale and (B) 5° C. and 50° C. compared to AVA shown in log NF50 scale, each vaccine was at a 1:16 dilution.

FIGS. 10A-B. A graph showing NF50 over time for lyophilized rPA at (A) 5° C. and 50° C. compared to AVA shown in linear NF50 scale and (B) 5° C. and 50° C. compared to AVA shown in log NF50 scale, each vaccine was at a 1:64 dilution.

FIGS. 11A-B. A graph showing the comparison of NF50 at (A) day 35 and (B) day 42 for lyophilized rPA at 5° C. and 50° C. and AVA, each vaccine was at a 1:4 dilution.

FIGS. 12A-B. A graph showing the comparison of NF50 at (A) day 35 and (B) day 42 for lyophilized rPA at 5° C. and 50° C. and AVA, each vaccine was at a 1:16 dilution.

FIGS. 13A-B. A graph showing the comparison of NF50 at day 35 (A) day 35 and (B) day 42 for lyophilized rPA at 5° C. and 50° C. and AVA, each vaccine was at a 1:64 dilution.

FIG. 14. A graph showing a comparison of the % MLA (microphage lysis assay) value for rPA liquid vaccine (Liq rPA F1) stored at one month versus lyophilized vaccines (LyoA, LyoB, and LyoC) stored at four months as a function of temperature.

FIGS. 15A-B. (A) A graph showing the relative drop in SEC % purity over reference control as a function of storage temperature of three rPA lyophilized formulations (LyoA, LyoB, and LyoC) stored for four months compared to liquid rPA stored for one month. (B) Shows a typical size exclusion chromatography (SEC-HPLC) chromatograph of rPA BDS reference standard.

FIGS. 16A-B. (A) A graph showing the relative drop in % AEX purity as a function of storage temperature of three rPA lyophilized formulations (LyoA, LyoB, and LyoC) stored for four months compared to liquid rPA stored for one month. (B) Shows an anion exchange chromatography (AEX-HPLC) chromatographs of rPA BDS reference standard.

FIGS. 17A-D. Graphs showing the comparison of NF50 at dose level 0.25 (Panel A), 0.125 (Panel B), 0.0625 (Panel C) and 0.03125 (Panel D) for LyoA stored at 5, 25, and 40° C. for one month.

FIGS. 18A-D. Graphs showing the comparison of NF50 at dose level 0.25 (Panel A), 0.125 (Panel B), 0.0625 (Panel C) and 0.03125 (Panel D) for LyoB stored at 5, 25, and 40° C. for one month.

FIGS. 19A-D. Graphs showing the comparison of NF50 at dose level 0.25 (Panel A), 0.125 (Panel B), 0.0625 (Panel C) and 0.03125 (Panel D) for LyoC stored at 5, 25, and 40° C. for one month.

FIGS. 20A-D. Graphs showing the comparison of NF50 at dose level 0.25 (Panel A), 0.125 (Panel B), 0.0625 (Panel C) and 0.03125 (Panel D) for liquid rPA stored at 5, 25, and 40° C. for one month.

FIG. 21. A graph showing NF50 values and the standard deviation of mean for 12 formulations as described in Example 8.

### DETAILED DESCRIPTION

For many years it has been believed that alum containing vaccines cannot be frozen. Accordingly, alum containing vaccines are not frozen or lyophilized (requires freezing), and alum-containing liquid vaccines are typically discarded if a break in the cold chain causes freezing. The inventors of the present invention made the exciting discovery that when a sugar such as trehalose or sucrose makes up about 20% (w/v) or more of an anthrax vaccine composition, the alum in the composition does not collapse as a result of freezing or thawing. Alum collapse is easy to identify and is associated with loss of vaccine potency and particle aggregation.

The inventors also identified additional stabilizing ingredients and process parameters that help prevent and reduce alum gel collapse. By adding amino acids to a formulation for instance, the amount of sugar required to prevent alum gel collapse can be reduced, e.g., to about 10% (w/v). Process changes that have a positive effect on alum gel height include freezing suspended particles (rather than settled particles) and increasing the freeze rate.

The section headings used herein are for organizational purposes only and are not to be construed as limiting the subject matter described. All documents, or portions of documents, cited herein, including but not limited to patents, patent applications, articles, books, and treatises, are hereby expressly incorporated by reference in their entirety for any purpose. In the event that one or more of the incorporated documents or portions of documents defines a term that contradicts that term's definition in the application, the definition that appears in this application controls.

The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components comprising more than one unit unless specifically stated otherwise. The word “about” means within about 1 unit.

As used herein, Protective Antigen (PA) or recombinant Protective Antigen (rPA) is the component of anthrax toxin (approx 83 kDa) that contains the receptor-binding and translocation domains. One example of a full length PA amino acid sequence is:

(SEQ ID NO: 1)
EVKQENRLLNESESSSQGLLGYYFSDLNFQAPMVVTSSTTGDLSIPSSEL
ENIPSENQYFQSAIWSGFIKVKKSDEYTFATSADNHVTMWVDDQEVINKA
SNSNKIRLEKGRLYQIKIQYQRENPTEKGLDFKLYWTDSQNKKEVISSDN
LQLPELKQKSSNSRKKRSTSAGPTVPDRDNDGIPDSLEVEGYTVDVKNKR
TFLSPWISNIHEKKGLTKYKSSPEKWSTASDPYSDFEKVTGRIDKNVSPE
ARHPLVAAYPIVHVDMENIILSKNEDQSTQNTDSQTRTISKNTSTSRTHT
SEVHGNAEVHASFFDIGGSVSAGFSNSNSSTVAIDHSLSLAGERTWAETM
GLNTADTARLNANIRYVNTGTAPIYNVLPTTSLVLGKNQTLATIKAKENQ
LSQILAPNNYYPSKNLAPIALNAQDDFSSTPITMNYNQFLELEKTKQLRL
DTDQVYGNIATYNFENGRVRVDTGSNWSEVLPQIQETTARIIFNGKDLNL
VERRIAAVNPSDPLETTKPDMTLKEALKIAFGFNEPNGNLQYQGKDITEF
DFNFDQQTSQNIKNQLAELNATNIYTVLDKIKLNAKMNILIRDKRFHYDR
NNIAVGADESVVKEAHREVINSSTEGLLLNIDKDIRKILSGYIVEIEDTE
GLKEVINDRYDMLNISSLRQDGKTFIDFKKYNDKLPLYISNPNYKVNVYA
VTKENTIINPSENGDTSTNGIKKILIFSKKGYEIG.

SEQ ID NO: 1 is the amino acid sequence of rPA102 which is expressed from plasmid pPA102. During secretion of rPA102 from B. anthracis ΔSterne-1(pPA102)CR4 into the extracellular space, the first 29 amino acids (the signal peptide) are removed yielding the mature rPA protein of 735 amino acids (82,674 Da). The mature rPA sequence is underlined (SEQ ID NO: 2).

The rPA102 amino acid sequence is but one example of one particular anthrax protein within the scope of the invention. Additional amino acid sequences of PA proteins, including native proteins, from various strains of anthrax are known in the art and include, for example, GenBank Accession Nos: NP_{—}652920.1, ZP_{—}02937261.1, ZP_{—}02900013.1, ZP_{—}02880951.1 which are incorporated by reference. Various fragments, mutations, and modifications in PA to reduce its toxicity or to improve its expression characteristics are also known, such as those described elsewhere in the specification, as are various fusion proteins. Those fragments, mutants, and fusion proteins are included in the term “PA” unless the context or text clearly indicates that those forms are excluded. Where indicated, PA fragments, mutants, and fusion proteins (whether with full length PA or a PA fragment) are those that elicit an antisera that is active in the toxin neutralization assay (TNA).

As used herein, “temperature stable,” “stable” or “stability” refers to the stability of the alum gel and potency of a vaccine after a freeze/thaw cycle. A stable vaccine as used herein is a vaccine that exhibits no or little decrease in activity and/or potency and/or alum gel collapse and/or particle aggregation after a freeze/thaw cycle as compared to a comparable liquid vaccine that is kept between 2°-8° C. Stability can be measured using any one or more of the assays described herein, including the working examples, as well as assays known in the art that are used to measure activity, potency and/or peptide degradation.

In certain embodiments, the immunogenicity of an antigen or vaccine, e.g., protective antigen, can be measured by calculating 50% neutralization factor (NF50). The geometric mean of the NF50 (GeoMean or <NF50>gm) for vaccine formulation can be calculated based on the NF50 values from a given number of data points. In certain embodiments, the NF50 and/or GeoMean is determined by using serum samples from a standard Toxin Neutralization Assay (TNA) (Hering et al., Biologicals 32 (2004) 17-27; Omland et al., Clinical and Vaccine Immunology (2008) 946-953; and Li et al., Journal of Immunological Methods (2008) 333:89-106), e.g., serum from immunized mice or rabbits. The dilution of serum resulting in 50% neutralization of toxin is the “ED50”. The neutralization capacity of each test serum in relation to that of a reference serum (50% neutralization factor, or NF50, also known as the neutralization ratio) is calculated from the quotient of the ED50 of the reference serum and the ED50 of the test serum, i.e., the neutralization factor, NF50 is calculated as follows:

$$NF 50 = \frac{ED {50}_{sample}}{ED {50}_{reference}}$$

In certain embodiments, a T-test or one-way ANOVA can be used to compare the geometric mean of NF50 from different formulation at the 95% confidence level. In one embodiment, if the p value of the GeoMean NF50 is larger than 0.05, there is no significant difference in NF50 among the formulations. In another embodiment, if the p value is less than 0.05, the geometric mean NF50 among the formulations is significantly different from each other.

Neutralization factor (NF50) calculations from mouse potency assay experiments show that the NF50 values and thus potency correlate with alum gel collapse. Accordingly, stability can be assessed by observing and measuring the alum gel of a vaccine that has been frozen overnight at −80° C. and then allowed to thaw at room temperature. A stable vaccine will exhibit little to no alum gel collapse as compared to a control vaccine (same composition but stored at 2°-8° C.). Alum gel height can be measured and a % difference between the frozen/thawed sample and 2°-8° C. control can be determined. In one embodiment, a difference of about less than 1%, 2%, 3%, 5%, 8%, 10% or 12% indicates a stable vaccine.

Stability can also be assessed by assaying the composition after freezing for intact protein (e.g., rPA intact with alum) or, conversely, desorbed protein (e.g., rPA desorbed from alum. For instance, stability can be determined by assaying and characterizing free rPA102 (release) by ELISA; protein structure by, for instance, differential scanning calorimetry and intrinsic fluorescence; desorbed free protein by A₂₈₀; purity and backbone degradation by SDS-PAGE, SEC and or RP-HPLC; charge variation by IEX or isoelectric focusing; and biochemical activity by microphage lysis assay (MLA).

In one embodiment, a temperature stable vaccine is a vaccine that after being exposed to freeze/thaw conditions (e.g., frozen vaccine or lyophilized vaccine), exhibits potency that is the same or at least about 98%, at least about 95%, at least about 93%, at least about 90%, at least about 88% or at least about 85% the same as a comparable liquid vaccine stored at about 2°-8° C. In one embodiment, an anthrax mouse potency assay is used to determine whether a frozen or lyophilized vaccine is potent.

In some embodiments, a composition retains at least 80%, at least 90% or at least 95% immunogenicity after storage in lyophilized form for at least 1 month at 40° C.

The vaccines of the invention are temperature stable vaccines. The temperatures over which a formulation of the invention is stable are generally below about 30° C., but may be above 30° C., 35° C., 40° C., 45° C., or 50° C. In some embodiments, the formulation's stability is in reference to a temperature below about 25° C., about 20° C., about 15° C., about 10° C., about 8° C., about 5° C., about 4° C., or about 2° C. Thus, in some embodiments, the temperature is in the range of about 25° C. to about −10° C., about 20° C. to about −10° C., about 15° C. to about −10° C., about 10° C. to about −10° C., about 8° C. to about −10° C., about 5° C. to about −10° C., about 15° C. to about −5° C., about 10° C. to about −5° C., about 8° C. to about −5° C., and about 5° C. to about −5° C.

The Examples section describes various methods for determining stability. In some embodiments, a vaccine of the invention shows no statistically significant decrease in stability after freeze thaw as compared to the same sample but fresh and/or stored 5° C. In some embodiments, a vaccine of the invention shows no statistically significant decrease in stability, immunogenicity, potency or any combination thereof after storage at −80° C., −20° C., 25° C., 40° C. and/or 50° C. for 1, 2, 3, 4, 5, 6, 9 12, 18, 24, 30, 36, 42, 48, 54 or 60 months as compared to storage at 5° C. for the same time period.

In some embodiments, the stability of a composition is measured by microphage lysis assay (MLA), size exclusion chromatography (SEC-HPLC) and/or anion exchange chromatography (AEX-HPLC).

In some embodiments, a composition retains at least 80%, at least 90% or at least 95% purity after storage in lyophilized form for at least 4 months at 50° C.

The vaccine compositions of the invention contain an antigen which is adsorbed to an aluminium adjuvant (alum) and an amount of sugar necessary to stabilize the formulation. For instance, the vaccine formulations disclosed herein exhibit little to no reduction in potency after exposure to freeze/thaw conditions when compared to a similar liquid vaccine that has been maintained at between 2°-8° C. and/or exhibit little to no collapse of alum gel.

The aluminium adjuvant (alum) can be, for instance, aluminium hydroxide, aluminium phosphate or aluminium sulphate. In one embodiment, the adjuvant is aluminium hydroxide (e.g., ALHYDROGEL). The amount of aluminium can vary quite a bit with apparently no effect on the stability of the alum gel (in other words, increasing the amount of alum in the composition does not appear to increase the likelihood that the alum gel will collapse). In one embodiment of the invention, the vaccine composition comprises about 1-10 mg/ml aluminium hydroxide. In another embodiment, the composition comprises about 1.5 to 5 mg/ml aluminium hydroxide. In another embodiment, the vaccine composition comprises about 1.5, 2, 2.5, 3, 3.5, 4, 4.5 or 5 mg/ml aluminium hydroxide.

It is believed that the stable vaccine compositions of the invention can be used to stabilize any antigen that is formulated with alum. For instance, the antigen can be a B. anthracis recombinant Protective Antigen (rPA) or a cell-free filtrate from an avirulent B. anthracis strain such as V770-NP1-R (e.g., anthrax vaccine adsorbed).

Methods of expressing B. anthracis proteins, including PA (as well as fragments, mutants, and fusion proteins) are described, for example in U.S. Pat. No. 7,201,912, to Park and Giri, U.S. Pat. No. 6,387,665 to Ivins et al., U.S. Pat. No. 6,316,006 to Worsham et al., and U.S. Pat. No. 7,261,900 to Leppla et al., each of which is incorporated by reference in its entirety. For example, as described in U.S. Pat. No. 7,201,912, pBP103 is an expression vector for full-length, wild-type rPA. The PA sequence from pBP103 is identical to that of wild-type PA.

Some embodiments of the invention include formulations comprising PA expressed in B. anthracis, including expression in either sporulating or non-sporulating strains of B. anthracis or both. For instance, the PA can be derived from non-sporulating B. anthracis strain ΔSterne-1 (pPA102)CR4 (i.e., rPA102). See, for instance, U.S. Pat. No. 6,316,006 and U.S. Pat. No. 6,387,665, both to Ivins et al., each of which is herein incorporated by reference in its entirety. Some compositions of the invention comprise a PA from the avirulent B. anthracis strain V770-NP1-R

The formulations of the invention may also include B. anthracis PA expressed by a heterologous organism. For instance, the invention includes PA expressed in E. coli.

In addition, various PA fragments, mutants, and fusion proteins have also been described and can be used in the current formulations. For example, PA may be modified to lack a functional binding site, thereby preventing PA from binding to either Anthrax Toxin Receptor (ATR) (see Bradley, K. A., Nature (2001) 414:225-229) to which native PA binds, or to native LF. By way of example, a modification made within or near to amino acid residues 315-735 or within or near to residues 596-735 of Domain 4 may render PA incapable of binding to ATR. Alternatively (or in addition), the PA furin cleavage site “RKKR” (SEQ ID NO: 3), which in most full length PA sequences is found at or around residues 163-168, may be inactivated by deletion, insertion, or substitution within or near to the furin cleavage site. For example, all of the furin cleavage site residues of native PA may be deleted. Other mutant PAs include those in which the dipeptide Phe-Phe has been modified to render the PA resistant to chymotrypsin. A PA fragment or PA fusion protein may also be a PA mutant.

Specific examples of PA fragments include those in U.S. Pat. No. 7,201,912, for example, PA64 expressed by pBP111, PA47 expressed by pBP113, PA27 expressed by pBP115. Some of those fragments may also include mutations to, for example, eliminate the furin cleavage site RKKR (SEQ ID NO: 3) or the chymotrypsin sensitive site formed by the dipeptide sequence Phe-Phe (FF). In addition, fragments may include one or two additional amino acids at the N-terminus. Examples of fusion proteins involving PA include those in U.S. Pat. No. 7,201,912, for example the PA-LF fusion proteins expressed by plasmids pBP107, pBP08, and pBP109. The invention also includes formulations comprising a HIS-tag PA. When a fragment, mutant, or fusion protein is used, however, it is generally desirable that the fragment, mutant, or fusion protein elicit protective immunity to a challenge with, e.g., an LD₅₀, of anthrax spores of the Ames strain in one or more of mice, guinea pigs, or rabbits.

PA from a recombinant source and/or a non-recombinant source can be used and the stability of such preparations improved by the formulations of the invention.

In one embodiment, the vaccine composition comprises about 75 to 750 μg/ml, 100 to 500 μg/ml, 100 to 250 μg/ml, 100 to 750 μg/ml or 250 to 750 μg/ml of antigen, e.g., rPA. For instance, the invention includes a vaccine comprising about 150, 200, 250, 300, 350, 400, 450 and 500 μg/ml of antigen, e.g., rPA. In some embodiments, the vaccine comprises approximately 175 μg antigen (e.g., rPA) per 1500 μg aluminum hydroxide. In some embodiments, the vaccine comprises approximately 200 μg/mL antigen (e.g., rPA) and about 0.5 mg/mL aluminum hydroxide. In further embodiments, the vaccine comprises approximately 250 μg antigen (e.g., rPA) per 100 to 250 μg aluminum hydroxide. In some embodiments, an antigen is an Anthrax antigen such as protective antigen. In some embodiments, a protective antigen is at least about 80% identity to the polypeptide of SEQ ID NO: 2. Some compositions of the invention comprise about 150-500 μg/ml protective antigen or about 150, 175, 200, 225, 250, 275, 300, 325, 400, 375, 400, 425, 450, 475 or 500 μg/ml protective antigen.

In some embodiments a composition of the invention contains about 0.5 to 1.5 mg/ml aluminum hydroxide. In some embodiments, a composition contains about 0.5 mg/ml or about 1.5 mg/ml aluminum hydroxide.

In some embodiments, an aluminum adjuvant is selected from the group consisting of aluminum hydroxide, aluminum phosphate and aluminum sulfate.

An anthrax vaccine of the invention, whether it be a vaccine comprising rPA or a cell-free filtrate from an avirulent B. anthracis strain, can be administered to a subject pre-exposure or post-exposure to B. anthracis. When administered post-exposure, the vaccine may be administered in conjunction with antibiotics.

In another embodiment, the antigen is a protein (e.g., recombinant) based antigen selected from the group consisting of hepatitis B protective antigens, Clostridium botulinum neurotoxin protein, Herpes Simplex Virus antigens, Influenza antigens, Congenital cytomegalovirus antigens, Tuberculosis antigens, HIV antigens, Diphtheria antigens, Tetanus antigens, Pertussis antigens, Staphylococcus enterotoxin B (SEB), and Yersinia pestis protective antigens and F1-V fusion protein. Antigens can be derived, for instance, from papillomavirus (e.g., HPV), influenza, a herpesvirus, a hepatitis virus (e.g., a hepatitis A virus, a hepatitis B virus, a hepatitis C virus), Meningococcus A, B and C, Haemophilus influenza type B (HIB), Helicobacter pylori, Vibrio cholerae, Streptococcus sp., Staphylococcus sp., Clostridium botulinum, Bacillus anthracis and Yersinia pestis.

The vaccines of the present invention can withstand freezing overnight at −80° C. with little to no loss of potency or collapse of alum gel. The invention includes frozen liquid vaccines as well as lyophilized vaccines (also referred to herein as freeze dried vaccines). As disclosed herein, the lyophilization process includes the freezing of a liquid composition. The frozen composition is then subjected to sublimation under freezing. For the lyophilized vaccines, the disclosed vaccine components and amounts refer to the amounts used in the liquid composition that is then subjected to freezing and not necessarily the dried lyophilized cake or reconstituted vaccine. The final lyophilized vaccine cake (a dry composition) may contain different percentages of components due to the drying process.

The present invention provides method for lyophilizing a vaccine comprising (i) freezing a composition of the invention and (ii) subjecting the frozen composition to sublimation.

In one embodiment, the vaccine of the invention comprises about 20% or more of a glass forming agent such as sugar. In one embodiment, the glass-forming agent is a reducing sugar. In one embodiment, the vaccine comprises a non-reducing sugar such as trehalose or sucrose. In one embodiment, the glass forming agent is trehalose or sucrose. If the vaccine is lyophilized, it may be preferable to use no more than about 40% sugar, prior to lyophilization, so that the vaccine forms a cake-like composition. The vaccine may comprise about 15%, 16%, 17%, 18%, 19%, 20%, 21%, 22%, 23%, 24%, 25%, 26%, 27%, 28%, 29%, 30%, 35% or 40% sugar, e.g., prior to lyophilization. In one embodiment, the vaccine composition comprises about 10-40%, 10-35%, 10-30%, 10-25%, 10-20%, 35-40%, 30-40%, 25-40%, 20-40%, 15-40%, 20-30%, 20-25%, 25-30%, 25-35%, 21-40%, 21-35%, 21-30% 21-25% or greater than 10%, 15%, 20%, 21%, 22%, 23%, 24%, 25%, 26%, 27%, 28%, 29%, 30%, 31%, 32%, 33%, 34%, 35%, or 36% (w/v) sugar, e.g., prior to lyophilization. In some embodiments, a composition contains greater than about 15%, 16%, 17%, 18%, 19%, 20%, 21%, 22%, 23%, 24% and 25% (w/v) sugar, e.g., prior to lyophilization.

As disclosed herein, the inventors of the present invention have identified that alum vaccine compositions comprising at least about 20% trehalose or sucrose can withstand freeze/thaw conditions. When certain additional stabilizing agents are added (e.g., a surfactant and/or amino acid) and/or the process improvements disclosed herein are incorporated (e.g., increasing freeze rate, freezing of suspended particles), the amount of sugar can be reduced to about 15% (w/v) or even about 10% (w/v) without affecting potency of the vaccine.

In one embodiment of the invention, a vaccine composition comprising an antigen adsorbed to an aluminium adjuvant and a sugar (e.g., 15% w/v or more) also contains a solubilizing agent such as a surfactant, e.g., prior to lyophilization. In one embodiment, the surfactant is a nonionic detergent such as polysorbate 80 (TWEEN® 80). In one embodiment, the vaccine composition comprises between about 0.001% and about 0.05% surfactant (such as polysorbate 80). In one embodiment, the composition comprises about 0.020%, about 0.025% or about 0.020% to 0.025% (w/v) surfactant (such as polysorbate 80). Other surfactants that can be used include, but are not limited to, polysorbate 20, pluronic L68, polyoxyethylene 9-10 nonyl phenol (Triton™ N-101, octoxylnol 9), Triton™ X-100, and sodium dexoycholte. In one embodiment, the surfactant is removed during the manufacturing process so that no surfactant is present in the final drug product. In one embodiment, a surfactant is present during freezing, e.g., of lyophilization process. In some embodiments, a formulation of the invention does not comprise a surfactant.

The inventors have found that the percentage of sugar may be reduced to as much as about 10% (w/v) with little to no effect on potency and/or little to no alum gel collapse if amino acids (for instance, alanine, arginine, glycine and proline) are added to the composition, e.g., prior to freezing and/or lyophilization. The amount of amino acid added to the vaccine composition can vary. In one embodiment, the vaccine composition comprises 0.5 to about 15% (w/v) of an amino acid or combination of amino acids. In one embodiment, the vaccine composition comprises about 2-10% (w/v) of an amino acid or combination of amino acids. In one embodiment of the invention, the vaccine comprises about 2% arginine or alanine. In another embodiment of the invention, the vaccine comprises about 10% glycine. Is some embodiments, a vaccine composition comprises about 2-10%, 2-8%, 2-6%, 2-4%, 2-3%, 3-10%, 5-10%, 7-10%, 2.5-5%, 3-5%, 3-7%, or 4-6% (w/v) of an amino acid or combination of amino acids. In some embodiments, two, three or more amino acids are present, such as selected from alanine, glycine, proline and/or arginine. In some embodiments, a composition contains about 0.5-4%, 1-4%, 1.5-4%, 2-4%, 2.5-4%, 3-4%, 3.5-4%, 0.5-1%, 0.5-1.5%, 0.5-2%, 0.5-2.5%, 0.5-3%, 0.5-3.5%, 0.5-4%, 1-3%, 1-2%, 2-3%, or 1.5-2.5% (w/v) alanine or arginine. In some embodiments, a composition contains about 2%, 1.75%, 2.25%, 2.5%, 2.75%, 3%, 3.25%, 3.5%, 3.75% or 4% (w/v) alanine or arginine.

In some embodiments, a composition contains about 6-12%, 7-12%, 8-12%, 9-12%, 10-12%, 11-12%, 6-11%, 6-10%, 6-9%, 6-8%, 6-7%, 7-11%, 8-10%, 7-10%, 11-9%, 7-8%, 8-9% or 9-10% (w/v) glycine. In some embodiment, a composition contains about 6%, 7%, 8%, 9%, 10%, 11% or 12% (w/v) glycine. In some embodiments, the recited concentration of amino acid is prior to freezing and/or lyophilization.

In some embodiments, a formulation does not comprise an amino acid(s) solution or does not contain an amino acid(s), other than the amino acids that are part of the polypeptide antigen.

In some embodiments, the formulation further comprises one or more additional ingredients. For example, the formulation may include one or more salts, such as sodium chloride, sodium phosphate, or a combination thereof. In general, each salt is present in the formulation at about 10 mM to about 200 mM.

The vaccine formulations may contain a buffer such as 20 mM TRIS-HCL. The pH of the formulation may also vary. In general, it is between about pH 6.2 to about pH 8.0. In one embodiment, the pH of the vaccine is about 7.4.

In another embodiment, the formulation further comprises a sugar alcohol such as sorbitol. In one embodiment, the formulation comprises 0.25% sorbitol.

In some embodiments, compositions and vaccine formulations of the invention may contain additional adjuvants, for instance, ImmunoStimulatory Sequences (ISS, CpG), and calcium phosphate. For ISS, protein samples are generally used at a final protein concentration 50 μg/ml. Other non-limiting examples of adjuvants include, but are not limited to: CGP7909 (e.g., see U.S. Pat. No. 7,223,741, which is herein incorporated by reference in its entirety), CpG1018 (see, for instance, US 2010/0183675, which is herein incorporated by reference in its entirety), Glucopyranosyl Lipid Adjuvant (GLA), PolyI PolyC (PIPC), N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19840A, referred to as MTP-PE), and RIBI, which contains three components extracted from bacteria, monophosphoryl lipid A, trehalose dimycolate and cell wall skeleton (MPL+TDM+CWS) in a 2% squalene/TWEEN 80 emulsion.

The invention includes compositions comprising the following formulations: 0.15 mg/mL antigen, 1.5 mg/mL aluminium, 20% trehalose, 2% alanine and 0.025% surfactant; 0.5 mg/mL antigen, 5 mg/mL aluminium, 20% trehalose, 2% alanine and 0.025% surfactant; 0.5 mg/mL antigen, 5 mg/mL aluminium, 20% trehalose, 1% sucrose, 2% alanine and 0.025% surfactant; and 0.5 mg/mL antigen, 5 mg/mL aluminium, 20% trehalose, 2% alanine and 0.025% surfactant. In some embodiments the antigens and/or surfactants in the these formulations are PA and Tween® 80, respectively. In some embodiments, a composition of the invention comprises 5 mM NaPi, pH 7.0 buffer or 20 mM Tris, pH 7.4. Other compositions included in the invention are described in the Examples.

Vaccines of the invention can be prepared for use as injectables. The composition can be a liquid formulation that is temperature stable (e.g., can withstand a freeze/thaw cycle) or a frozen composition. The composition may also be used to produce a lyophilized dry powder vaccine which can be reconstituted, e.g., with a pharmaceutically acceptable carrier prior to administration. Vaccine administration is generally by conventional routes, for instance, intravenous, subcutaneous, intraperitoneal, or mucosal routes. The administration may be by parenteral injection, for example, a subcutaneous or intramuscular injection.

In some embodiments, a composition of the invention is subjected to freezing and followed by sublimation under vacuum to produce a lyophilized composition.

The term “reconstituted” or “reconstitution” refers to the restoration of a lyophilized form to a liquid form, e.g., by rehydration, of a substance previously altered for preservation and/or storage, e.g., the restoration to a liquid state of a lyophilized rPA formulation of the application that has been stored. A lyophilized composition of the present application can be reconstituted in any aqueous solution which produces a stable, aqueous solution suitable for administration. Such an aqueous solution includes, but is not limited to, sterile water, TE (Tris EDTA), phosphate buffered saline (PBS), Tris buffer or normal saline. A lyophilized sample can be reconstituted with a lower, the same or higher volume than was used to lyophilize the sample.

It should be understood that a dose of a reconstituted lyophilized vaccine formulation of the application can be determined in light of various relevant factors including the conditions to be treated, the chosen route of administration, the age, sex and body weight of the individual patient, and the severity of the patient's symptom, and can be administrated in a single dose, divided dose or multiple doses.

The vaccines are administered in a manner compatible with the dosage formulation, and in such amount as will be prophylactically and/or therapeutically effective. The quantity to be administered, which is generally in the range of 5 μg to 500 μg of antigen per dose, depends on the subject to be treated, capacity of the subject's immune system to synthesize antibodies, and the degree of protection desired. In one embodiment, the vaccine comprises at least about 10 μg PA, 25 μg PA, 50 μg PA, 75 μg PA, 100 μg PA, 125 μg PA, 150 μg PA, 200 μg PA, 225 μg PA, 250 μg, 275 μg, 300 μg PA. Precise amounts of antigen are dependent on the antigen to be delivered.

The vaccine may be given in a single dose schedule, or optionally in a multiple dose schedule. The vaccine composition may be administered, for instance, in a 0.5 mL dose. For pre-exposure prophylaxis, a multiple dose schedule is one in which a primary course of vaccination may be with 1-6 separate doses, followed by other doses given at subsequent time intervals to maintain and or reinforce the immune response, for example, at 1-4 months for a second dose, and if needed, a subsequent dose(s) after several months.

For post-exposure prophylaxis, the vaccine may also be administered according to a single dose or multiple dose regimen. For instance, in one embodiment, the vaccine is administered in 3 doses at times 0, 2 and 4 weeks post exposure. The dosage regimen will also, at least in part, be determined by the need of the individual, upon the judgment of the practitioner and/or upon results of testing, e.g., measuring levels of immune response to the vaccine/antigen(s) such as antibody levels and/or T-cell activity against the antigen(s).

In addition, the vaccine containing the immunogenic antigen(s) may be administered in conjunction with other immunoregulatory agents, for example, immunoglobulins, antibiotics, interleukins (e.g., IL-2, IL-12), and/or cytokines (e.g., IFN-beta, IFN-alpha).

In one embodiment, the vaccine is administered to a subject post-exposure to anthrax. In this embodiment, the vaccine may be administered in conjunction with an antibiotic. Antibiotics that may be administered with the vaccine include, but are not limited to, penicillin, doxycycline and ciprofloxacin.

The invention includes methods of treating (post-exposure prophylaxis) or preventing (pre-exposure prophylaxis) an anthrax infection comprising administering to a subject a pharmaceutically effective amount of a vaccine of the invention. In one embodiment, the anthrax infection is the result of inhaling anthrax (inhalation anthrax). As used herein, a pharmaceutically effective amount of a vaccine is an amount that induces an immune response. In one embodiment, a pharmaceutically effective amount of a vaccine is an amount comprising at least 25 μg PA. As used herein, a subject is a mammal such as a human.

The invention also provides methods of stimulating an immune response in a subject by administering to the subject an amount of a vaccine of the invention sufficient to stimulate an immune response. In one embodiment, immune stimulation is measured by increases in antibody titer that is specific for the antigen in the vaccine. In still other embodiments, immune stimulation is measured by an increased frequency in cytotoxic T lymphocytes specific for the antigen in the vaccine.

Also provided are methods of vaccinating a subject against a pathogen comprising administering a composition of the invention. Additionally provided are methods of vaccinating a subject against a pathogen comprising administering to a subject a pharmaceutical composition reconstituted from a lyophilized composition of the invention. The invention further includes methods of producing potent, alum based frozen vaccines comprising suspending a composition comprising at least about 10%, at least about 15%, at least about 20%, at least about 21%, at least about 25% or at least about 30% sugar and an antigen adsorbed to an aluminum adjuvant and freezing said composition at a rate sufficient to freeze the suspended composition before sedimentation occurs, e.g., flash freezing.

Some embodiments of the invention provide methods of preparing a stable lyophilized composition, comprising lyophilizing a composition of the invention, wherein the stability of the reconstituted lyophilized composition is measured by microphage lysis assay (MLA), size exclusion chromatography (SEC-HPLC) and/or anion exchange chromatography (AEX-HPLC).

For anthrax vaccines, the immunogenicity of the formulations can be tested as described in the various examples. For example, mice can be immunized with, for example, 10 μg, 20 g, or more of rPA suspended in an adjuvant emulsion. Control mice are immunized with saline emulsified in adjuvant for use as negative controls. The mice are generally immunized, then bled at various intervals, e.g., day 0, day 21 and day 28 post-immunization. The serum is then analyzed for the presence of specific antibody, e.g., by ELISA, which can also be used to determine the titer of the antisera.

A mouse toxin-neutralizing antibody assay can also be used to determine if the anthrax vaccine formulations elicit protective antibodies. In this assay, mice immunized with rPA are then challenged i.p. with 2 lethal doses of lethal toxin (PA and lethal factor (LF)). Four days after challenge, the mice are scored for survivors.

The rPA formulations can also be used to prepare compositions comprising neutralizing antibodies that immunoreact with the anthrax toxin. The resulting antisera can be used for the manufacture of a medicament for treating exposure to anthrax. In one embodiment of the invention, the antibody composition comprises a purified anti-PA antibody. By “purified,” it is meant that the antibody is substantially free of other biological material with which it is naturally associated. Purified antibodies of the invention are at least 60% weight pure, at least 70% weight pure, at least 80% weight pure, at least 90% weight pure or at least 95% weight pure. The antisera, or antibodies purified from the antisera, can also be used as diagnostic agents to detect either PA fragments or native protein.

The frozen and lyophilized formulations of the invention can be manufactured with increased potency by increasing the freeze rate. In one embodiment the formulation is flash frozen.

Potency can also be increased by freezing suspended rather than settled compositions. Compositions can be suspended by gentle shaking and immediately frozen.

The invention will be further clarified by the following examples, which are intended to be purely exemplary of the invention and in no way limiting.

### Example 1

### Freeze/Thaw of Liquid rPA and AVA Vaccines with and without Trehalose

rPA102 vaccine formulations with and without trehalose were prepared as outlined in Table 1 below.

TABLE 1
Trehalose Formulations for Freeze/Thaw Assay

| | rPA | ALHYDROGEL | | | | | TWEEN |
|---|---|---|---|---|---|---|---|
| Sample | (mg/ml) | (mg/ml) | Buffer | pH | Trehalose | Arginine | 80 |
| rPA Control 1 | 0.15 | 1.5 | 20 mM | 7.4 | — | — | — |
| rPA Test 1 | | | TRIS- | | 20% | 2% | 0.025% |
| | | | HCl | | | | |

After compounding, each sample was divided into two 8 ml aliquots in 10 ml glass tubes. For each sample, after gentle mixing overnight, one tube was placed at −80° C. and the other tube was placed at 2-8° C. after gentle mixing overnight.

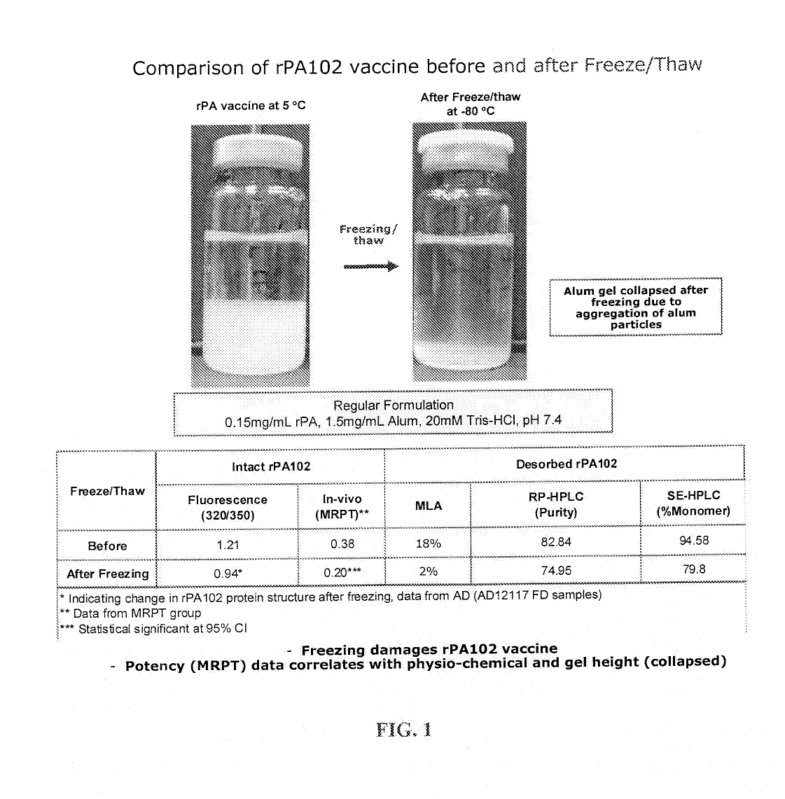

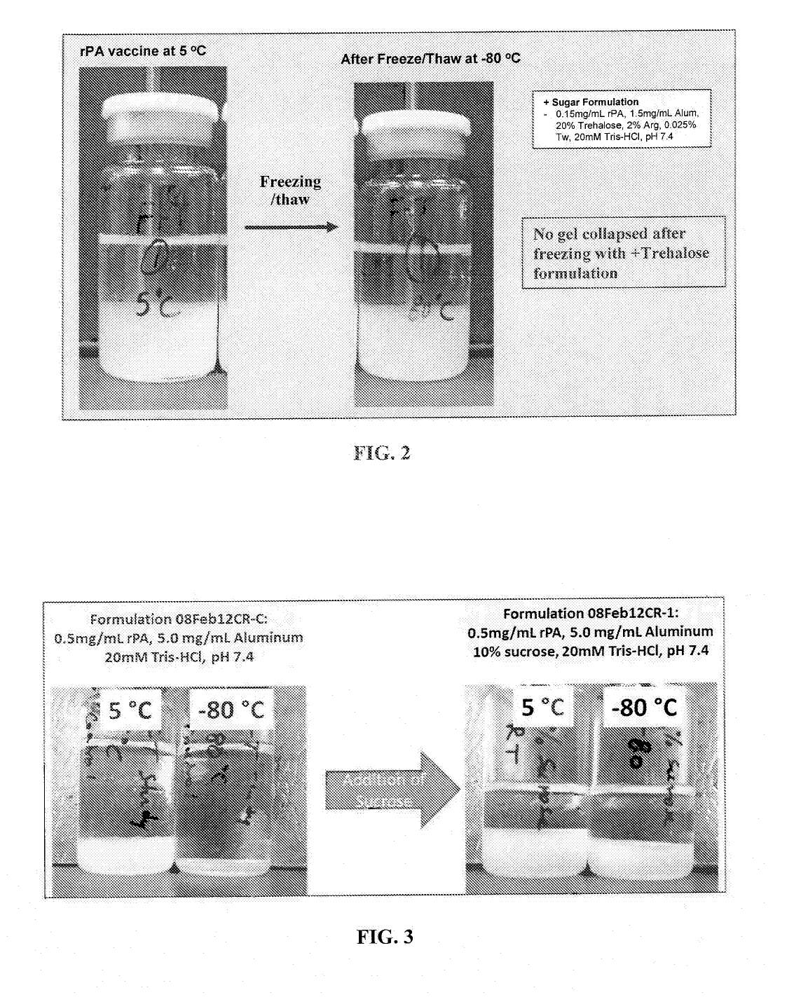

Samples stored at −80° C. overnight were thawed on the lab bench the next day for several hours (>3-4 hours) before observation and comparison to the 2-8° C. samples that were brought to room temperature. Samples were photographed and total liquid height and ALHYDROGEL (aluminum hydroxide) height were measured. Regular rPA102 vaccine was compared before and after freeze/thaw. FIG. 1 is a photograph comparing rPA Control 1 sample at 2-8° C. (labeled 5° C.) to the −80° C. sample after thaw. The photograph shows significant collapse of the alum gel in the rPA Control 1 sample subjected to freeze/thaw conditions. The level of sugar in a regular formulation that protected rPA102 from freeze/thaw stress was tested. As shown in FIG. 1, freezing damaged rPA102 vaccine, and the potency (MRPT) data correlated with physio-chemical and gel height (collapsed). FIG. 2 is a photograph comparing rPA Test 1 sample at 2-8° C. (labeled 5° C.) to the −80° C. sample after thaw. There is no noticeable collapse of the alum in the thawed rPA Test 1 sample. Table 2 provides an overview of the relative % alum height.

TABLE 2
Relative % Alum Height

| Formulation | 5° C. | −80° C. |
|---|---|---|
| rPA Control 1 | 100% | 32.6% |
| rPA Test 1 | 100% | 101.5% |

A similar freeze/thaw experiment was performed with a test composition containing 15% trehalose, 0.15 mg rPA/mL, 2% alanine, 0.025% polysorbate 80, 25 mM NaPi, pH 7.4 compared to a control formulation without 15% trehalose with similar result (data not shown).

A vial of BioThrax® (Anthrax Vaccine Adsorbed), AVA, and a vial AVA+25% trehalose were placed in −80° C. after gentle mixing. A second vial of BioThrax and a second vial of AVA+25% trehalose were placed at 2°-8° C. overnight after gentle mixing. The next day, the −80° C. vials were allowed to thaw and all vials were inspected. The aluminum gel height appeared to be about the same for the BioThrax stored at 2°-8° C. and the two AVA samples containing 25% trehalose. The aluminum gel height was much lower for BioThrax stored at −80° C. (no trehalose). (Data not shown).

### Example 2

### Freeze/Thaw of Liquid rPA Vaccines with and without Sucrose

rPA102 vaccine formulations with and without sucrose were prepared as outlined in Table 3 below.

TABLE 3
Sucrose Formulations for Freeze/Thaw Assay

| | | ALHYDROGEL | | | |
|---|---|---|---|---|---|
| Sample | rPA (mg/ml) | (mg/ml) | Buffer | pH | Sucrose |
| rPA Control 2 | 0.5 | 5 | 20 mM | 7.4 | — |
| rPA Test 2 | | | TRIS- | | 10% |
| | | | HCL | | |

After compounding, each sample was divided in to two 10 ml aliquots in 10 ml glass tubes. For each sample, one tube was placed at −80° C. after gentle mixing overnight and the other tube was placed at 2-8° C. after gentle mixing overnight.

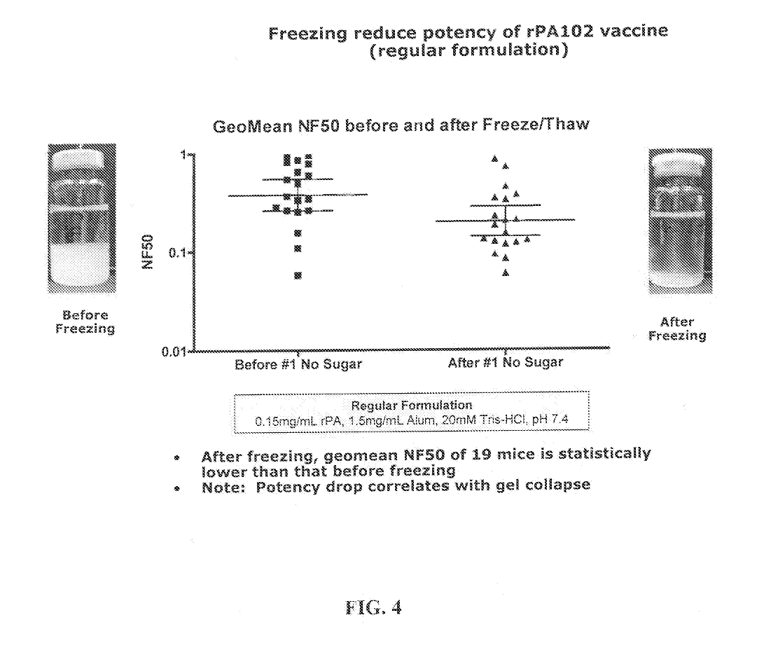

Samples stored at −80° C. overnight were thawed on the lab bench the next day for 2-3 hours before observations were made. FIG. 3 contains a photograph of each formulation from 2-8° C. (labeled 5° C.) and −80° C. after both being brought to room temperature. As shown, gel collapse occurred in both −80° C. samples (rPA Control 2 and rPA Test 2) after thaw as compared to the samples that remained refrigerated at 2-8° C. However, the amount of gel collapse was visibly greater in the rPA Control 2 sample that did not include sucrose.

### Example 3

### In Vivo Mouse Potency Assay

Lyophilized vaccines were prepared as outlined in Table 4. Dried vaccines were reconstituted with water for injection to a final rPA concentration of 0.15 mg/ml (75 μg/0.5 ml dose) and then diluted in normal saline by 10-fold to yield a dose level of 0.1 (DL).

Female CD-1 mice at 5-8 weeks of age and weighing 20-25 grams each were used for this study. The 0.1 DL of the vaccine was injected (0.5 ml) IP into groups of 20 female CD-1 mice and sera were collected on day 28 for the assessment of their ability to neutralize anthrax LT cytotoxicity in the toxin neutralization assay (TNA) in mice.

TABLE 4
Lyophilized Formulations for Mouse Potency Assay

| Samples | Formulation |
|---|---|
| Lyoph- | 10% trehalose, 0.5 mg/ml rPA, 5.0 mg/ml aluminum, 0.25% |
| ilized #1 | sorbitol, 75 mM NaCl, 1% arginine, 20 mM Tris-HCL, pH 7 |
| Lyoph- | No sugar, 0.15 mg/ml rPA, 1.5 mg/ml aluminum, 20 mM |
| ilized #2 | Tris-HCL, pH 7.4 |
| Lyoph- | 30% trehalose, 0.15 mg/ml rPA, 1.5 mg/ml aluminum, 2% |
| ilized #3 | arginine, 0.025% polysorbate 80, 20 mM Tris-HCl, pH 7.4 |
| Lyoph- | 20% trehalose, 0.15 mg/ml rPA, 1.5 mg/ml aluminum, 10% |
| ilized #4 | glycine, 0.025% polysorbate 80, 20 mM Tris-HCl, pH 7.4 |

The immunogenicity of the rPA102 formulation was investigated by calculating Neutralization factor (NF50). The neutralization factor, NF50 is defined as follows:

$$NF 50 = \frac{ED {50}_{sample}}{ED {50}_{reference}}$$

Where effective dose 50% (ED50) reference standard was prepared by using a qualified serum reference standard stored at or below −20° C.

The geometric mean (GeoMean) of the NF50 of each vaccine formulation was calculated based on 20 NF50 values from 20 mice. T-test or one-way ANOVA were used to compare the geometric mean of NF50 of each formulation at the 95% confidence level. If the p value of the geometric mean NF50 was larger than 0.05, there was no significant difference in NF50 (potency) among the formulations. If the p value was less than 0.05, the geometric mean NF50 of the formulations was significantly different from each other.

The control (lyophilized #2) was an rPA102 formulation without stabilizer (0.15 mg/ml rPA, 1.5 mg/ml aluminum, 20 mM Tris-HCL, pH 7.4). The effect of freezing on the GeoMean NF50 values from the relative mouse potency assay for the regular rPA102 formulation is shown in FIG. 4. The control formulation was susceptible to freeze/thaw damage with immunogenicity dropping significantly after the freezing process (FIG. 4). The drop in immunogenicity corresponded to the decrease in the height of the aluminum gel in the solution.

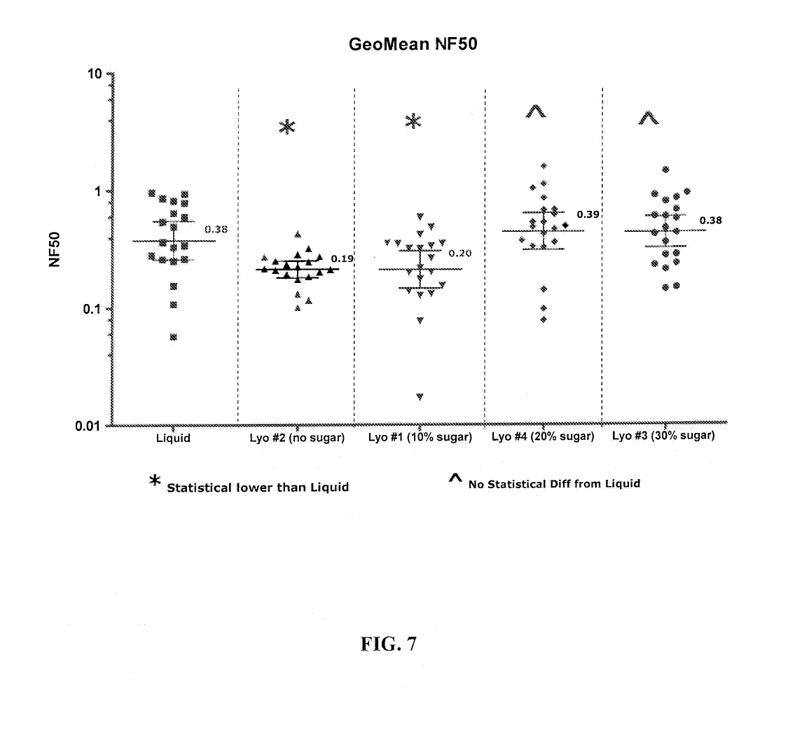

Lyophilized Samples #1 and 2 showed significantly lower immunogenic potency relative to Lyophilized Samples #3 and 4 (which contained 30% and 20% trehalose, respectively). The effect of sugar in the formulation on lyophilization of rPA102 vaccine was shown by GeoMean NF50 results in FIG. 7. Lyophilized Sample #1 (10% sugar) was not able to protect rPA102 from freeze-dry stress (see FIG. 7). These results showed that 20% and 30% sugar was able to protect rPA102 from lyophilization stress. The appearance of gel collapse correlated with potency loss.

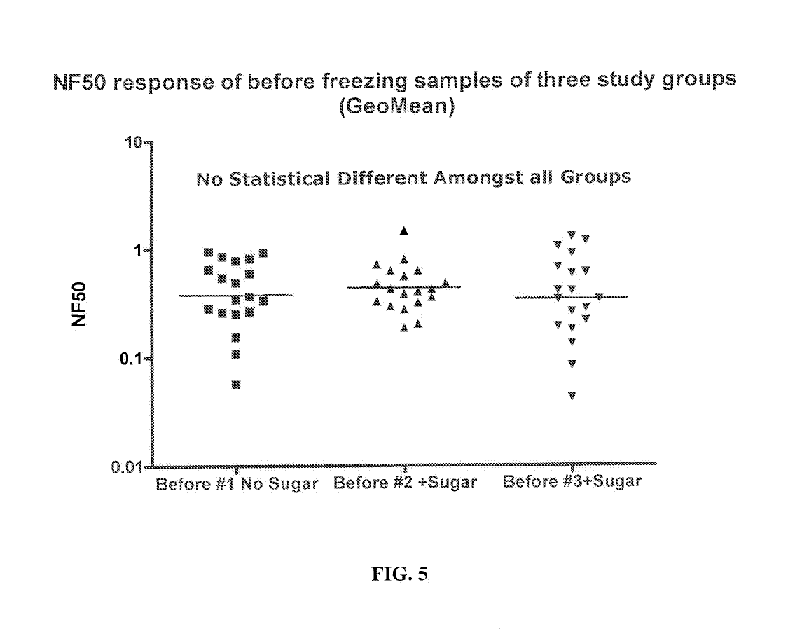

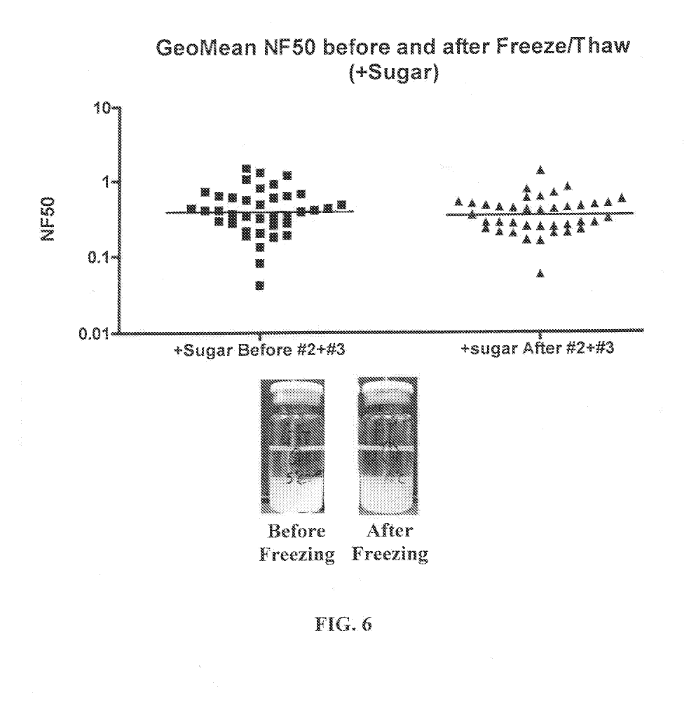

NF50 responses were determined from two more formulations that differ only by the absence or presence of trehalose: #1) 0.15 mg/mL rPA, 1.5 mg/mL alum, 2% arginine, 0.025% TWEEN 80 (polysorbate 80), 20 mM Tris-HCL, pH 7.4 and #2 & #3) 0.15 mg/mL rPA, 1.5 mg/mL alum, 20% trehalose, 2% arginine, 0.025% TWEEN 80 (polysorbate 80), 20 mM Tris-HCL, pH 7.4 and the effect of sugar before freezing on the GeoMean values for rPA102 is shown in FIG. 5. In FIG. 5 formulation groups #2 & #3 are just different vials of the same formulation. Adding trehalose had no impact on the immunogenicity of the formulation as demonstrated by no statistical change in NF50 for the two formulations (3 samples), with and without sugar and no freezing (FIG. 5). A comparison of GeoMean values before and after freezing of these trehalose containing formulations is shown in FIG. 6. There was no statistically significant change in immunogenicity (NF50) before and after freezing when this formulation was used (FIG. 6). In addition, there was no collapse of the alum gel (gel height maintained) after freezing with this formulation (photographs in FIG. 6). These results show that the tested formulations with 20% trehalose protected this rPA vaccine from freeze/thaw stress.

### Example 4

### Rabbit Immunogenicity and Stability Study

The immunogenicity of recombinant protective antigen (rPA) lyophilized vaccine formulations stored at 5 and 50° C. for 4 month was compared to Anthrax Vaccine Adsorbed (AVA) (BioThrax®) using a toxin neutralizing antibody assay (TNA) in New Zealand White (NZW) rabbits. This rabbit immunogenicity study utilized a two immunization schedule (Day 0 and Day 28) and bleeds were taken on days −1 or 0, 14, 21, 28, 35, 42, 56 and 70.

The rPA lyophilized vaccine was prepared using the ingredients shown in Table 5. The ingredients of the final formulation were blended prior to lyophilization and after reconstitution as shown in Table 5. Briefly, 2 mL of the suspension was filled in a 10 mL glass vial. The lyophilization was performed using a VirTis AdVantage lyophilizer. After lyophilization, the vaccines were stored at 5 and 50° C.

TABLE 5
rPA Formulation

| | Pre-lyophilization | Post reconstitution by |
|---|---|---|
| | (2 mL suspension | adding 6.11 mL water to |
| Ingredients | in 10 vial) | final volume of 6.67 mL |
| rPA, mg/mL | 0.5 | 0.15 |
| Aluminum, mg/mL | 5 | 1.50 |
| % Trehalose | 25% | 7.5% |
| Sorbitol | 0.25% | 0.075% |
| TWEEN 80 | 0.03% | 0.0075% |
| Arginine | 1% | 0.30% |
| NaCl, mM | 75 | 22.5 |
| Tris-HCl, mM pH 7.4 | 20 | 6.0 |
| Volume, mL | 2 | 6.67 |

In particular, stock solutions were prepared and (except for TWEEN 80 and NaCl) pH adjusted to 7.4 using 0.1N NaOH and/or 0.1N HCl. After stock solutions were prepared, 150 mL of the following formulation blend was prepared in a 200 mL Nalgene bottle: 0.5 mg/mL rPA, 5.0 mg/mL aluminum, 30% Trehalose (w/v), 0.25% Sorbitol (w/v), 1% Arginine (w/v), 0.025% TWEEN 80, 75 mm NaCl, 20 mM Tris-HCl, pH 7.4, which was used to fill 10 mL vials with 2 mL of formulation blend.

After filling vials, the samples were dried using a VirTis AdVantage lyophilizer with the following program:

### Initial Freezing

| Step | Process | Setting |
|---|---|---|
| Freeze | Freeze (° C.) | −60 |
| | Additional Freeze time (min) | 0 |
| | Condenser (° C.) | −80 |
| | Vacuum (mTorr) | 90 |

### Drying

| | Temp | Time | | Vac |
|---|---|---|---|---|
| Step | ° C. | (min) | R/H | mTorr |
| 1 | −60 | 120 | H | 90 |
| 2 | −28 | 60 | R | |
| 3 | −28 | 1250 | H | |
| 4 | −28 | 550 | H | |
| 5 | 25 | 480 | R | |
| 6 | 25 | 600 | H | |
| 7 | 30 | 120 | R | |
| 8 | 30 | 300 | H | |
| 9 | 35 | 120 | R | |
| 10 | 35 | 300 | H | |
| 11 | 40 | 120 | R | |
| 12 | 40 | 300 | H | |
| 13 | 45 | 120 | R | |
| 14 | 45 | 255 | H | |

### Post-Drying

| | |
|---|---|
| Secondary Dry Set-Point Temperature | +65° C. |

Post-Heat Settings

| | |
|---|---|
| Temperature (° C.) | +25 |
| Time (min) | 1250 |
| Vacuum (mTorr) | 1250 |

Vials were stored as described in Table 6. On the day of immunization, 6.11 mL sterile water for injection was added to each vial to reconstitute the lyophilized samples. The vials were mixed end over end until all formulation components were completely dissolved. Dilutions (1:4, 1:16 and 1:64) of each test and control article were prepared in sterile normal saline.

NZW rabbits were used for the present study. NZW rabbits are commonly used as an animal model for Bacillus anthracis disease to test for toxicity, immunogenicity and efficacy studies, and NZW rabbits are considered to be a well-characterized model since they have similar pathogenesis and clinical presentation as seen in humans (E K Leffel et al., Clin Vaccine Immunol. 19(18):1158-1164, 2012; A J Phipps et al., Microbiol Mol Biol Rev. 68(4):617-29, 2004). Each group of NZW rabbit (10 per vaccine group) received a 0.5 mL intramuscular injection with the 1:4, 1:16 & 1:64 dilutions of a lyophilized rPA vaccine formulation or AVA on days 0 and 28. AVA (BioThrax®) is a liquid anthrax vaccine that includes the 83 kDa protective antigen protein and is formulated with 1.2 mg/mL aluminum (added as aluminum hydroxide in 0.85% sodium chloride), 25 mg/mL benzethonium chloride and 100 mg/mL formaldehyde (added as preservatives).

Serum samples were collected at days −1 or 0, 14, 21, 28, 35, 42, 56 and prior to termination on day 70. The TNA assay was performed by using serum collected on day −1 or 0, 14, 35 and 42. Table 6 summarizes the study design.

TABLE 6
Rabbit Immunogenicity Study Design

| | | | Immunization | Dose | | # of |
|---|---|---|---|---|---|---|
| Group | Tested | Vaccine | Schedule | Volume | | Animals |
| # | Vaccine | Dilution | (Study Days) | (mL) | Blood Collection* | (Rabbits) |
| 1 | AVA | 1:4 | Day 0 and | 0.5 mL | Prior to the day of | 10 |
| 2 | (positive | 1:16 | 28 | | dose initiation | (5M/5F) |
| 3 | control) | 1:64 | | | (Day −1 or 0) and | |
| 10 | rPA | 1:4 | | | on Days 14, 21, 28, | |
| 11 | Lyophilization | 1:16 | | | 35, 42, 56 and 70 | |
| 12 | 5° C., 4 months | 1:64 | | | | |
| 13 | rPA | 1:4 | | | | |
| 14 | Lyophilization | 1:16 | | | | |
| 15 | 50° C., 4 months | 1:64 | | | | |

*Serum from Days −1 or 0, 14, 35 and 42 tested

The TNA assay is a functional test that evaluates the amount of antibody needed to inactivate the lethal B. anthracis toxin complex of LF and PA (lethal toxin, LT). The ability of test serum samples to neutralize lethal toxin in vitro was compared with that of a standard serum sample by using cytotoxicity as the endpoint of the assay (P R Pittman et al., Vaccine 24(17):3654-60, 2006).

Briefly, J774A.1 cells were cultured in flasks for 48 to 72 h in Dulbecco's modified Eagle media (DMEM) containing 4.5 g/liter d-glucose and supplemented with 10% heat-inactivated bovine serum, 2 mM L-glutamine, 1 mM sodium pyruvate, penicillin (50 U/ml), streptomycin (50 μg/ml), and 0.11 mM sodium bicarbonate. Cells were harvested and seeded in 96-well tissue culture plates at 30,000 cells/well, followed by 16 to 24 hour incubation. Serum samples were prepared in a separate 96-well microtiter plate at 2-fold dilutions for a total of seven dilutions per sample. The serum samples were then incubated with a constant concentration of LT (100 ng/ml PA and 80 ng/ml LF) for 1 hour. Then the serum sample with LT was added to the corresponding wells of the tissue culture plate containing the cells and incubated for four hours, after which 25 μl/well of 5 mg/ml of a tetrazolium salt, 3-(4,5-dimethylthiazolyl-2)-2,5-diphenyltetrazolium bromide (MTT), was added. After a 1-hour incubation, the cells were lysed by using 100 μl/well of acidified isopropanol (50% N, N-Dimethylformamide (with deionized water) and 20% SDS (200 g in 1 liter of 50% dimethylformamide)) with the pH adjusted to 4.7 using HCl. Assay plates were incubated for an additional 16-24 hour, absorbance was measured at 570 and 690 nm in which 690 optical density values were subtracted from 570 values using a calibrated Molecular Devices VersaMax Plate Reader. The ED50, which is the dose that produces a quantal effect in 50% of the population in optical density measurement, was determined using SoftMax Pro software (version 5.4.1, Sunnyvale, Calif.).

ED50 for the mouse reference serum (Lot #MS011211) was used to determine the NF50 values of the test serum samples and the positive control (NF50=ED50 Test/ED50 reference). The mouse reference standard was prepared by immunizing 300 mice with AVA vaccine containing CPG 7909 adjuvant. The serum from the 300 mice was collected, pooled and stored frozen at −80° C. as the reference standard. Tables 7A-C show a comparison of the average kinetic data (NF50) for lyophilized rPA stored at 5° C., lyophilized rPA stored at 50° C., and AVA control stored at 5° C. from serum samples at 14, 35 and 42 days, respectively.

TABLE 7A
Comparison of average kinetic data (NF50) at Day 14

| | Sample Storage Dilution | | | | | | | | |
|---|---|---|---|---|---|---|---|---|---|
| | rPA lyo | rPA lyo | AVA | rPA lyo | rPA lyo | AVA | rPA lyo | rPA lyo | AVA |
| | 5° C. | 50° C. | 5° C. | 5° C. | 50° C. | 5° C. | 5° C. | 50° C. | 5° C. |
| | 1:4 | 1:4 | 1:4 | 1:16 | 1:16 | 1:16 | 1:64 | 1:64 | 1:64 |
| Avg | 0.10 | 0.13 | 0.05 | NRS | NRS | NRS | 0.02 | 0.02 | NRS |
| Stdev | 0.07 | 0.08 | 0.02 | NRS | NRS | NRS | 0.01 | 0.01 | NRS |
| CV % | 0.72 | 61% | 50% | NRS | NRS | NRS | 31% | 26% | NRS |
| GeoMean | 0.07 | 0.11 | 0.04 | NRS | NRS | NRS | 0.02 | 0.02 | NRS |

NRS = Non-responders

TABLE 7B
Comparison of average kinetic data (NF50) at Day 35

| | Sample Storage Dilution | | | | | | | | |
|---|---|---|---|---|---|---|---|---|---|
| | rPA lyo | rPA lyo | AVA | rPA lyo | rPA lyo | AVA | rPA lyo | rPA lyo | AVA |
| | 5° C. | 50° C. | 5° C. | 5° C. | 50° C. | 5° C. | 5° C. | 50° C. | 5° C. |
| | 1:4 | 1:4 | 1:4 | 1:16 | 1:16 | 1:16 | 1:64 | 1:64 | 1:64 |
| Avg | 3.69 | 3.25 | 1.80 | 1.86 | 1.28 | 0.79 | 0.26 | 0.14 | 0.06 |
| Stdev | 2.19 | 1.47 | 0.85 | 0.82 | 0.99 | 0.46 | 0.25 | 0.11 | NRS |
| CV % | 59% | 45% | 47% | 44% | 78% | 59% | 96% | 77% | 0% |
| GeoMean | 3.14 | 2.98 | 1.61 | 1.70 | 0.94 | 0.67 | 0.16 | 0.10 | 0.06 |

TABLE 7C
Comparison of average kinetic data (NF50) at Day 42

| | Sample Storage Dilution | | | | | | | | |
|---|---|---|---|---|---|---|---|---|---|
| | rPA lyo | rPA lyo | AVA | rPA lyo | rPA lyo | AVA | rPA lyo | rPA lyo | AVA |
| | 5° C. | 50° C. | 5° C. | 5° C. | 50° C. | 5° C. | 5° C. | 50° C. | 5° C. |
| | 1:4 | 1:4 | 1:4 | 1:16 | 1:16 | 1:16 | 1:64 | 1:64 | 1:64 |
| Avg | 2.54 | 2.35 | 1.15 | 1.21 | 0.78 | 0.46 | 0.17 | 0.12 | 0.05 |
| Stdev | 1.74 | 1.18 | 0.60 | 0.67 | 0.60 | 0.26 | 0.17 | 0.08 | 0.04 |
| CV % | 68% | 50% | 52% | 56% | 77% | 56% | 97% | 64% | 75% |
| GeoMean | 2.07 | 2.13 | 1.01 | 1.08 | 0.57 | 0.40 | 0.11 | 0.10 | 0.04 |

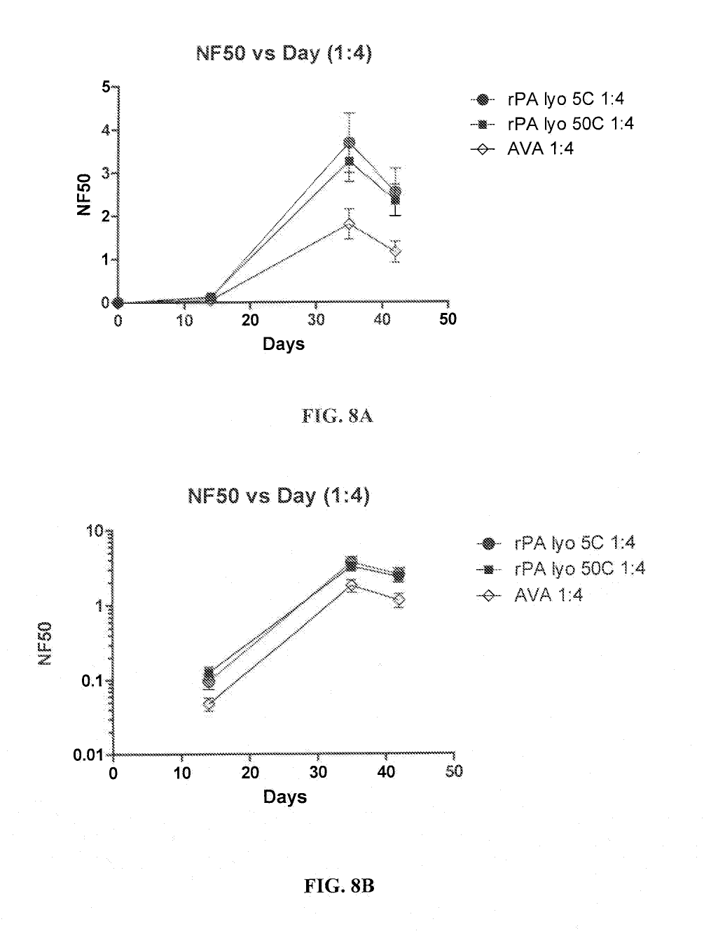

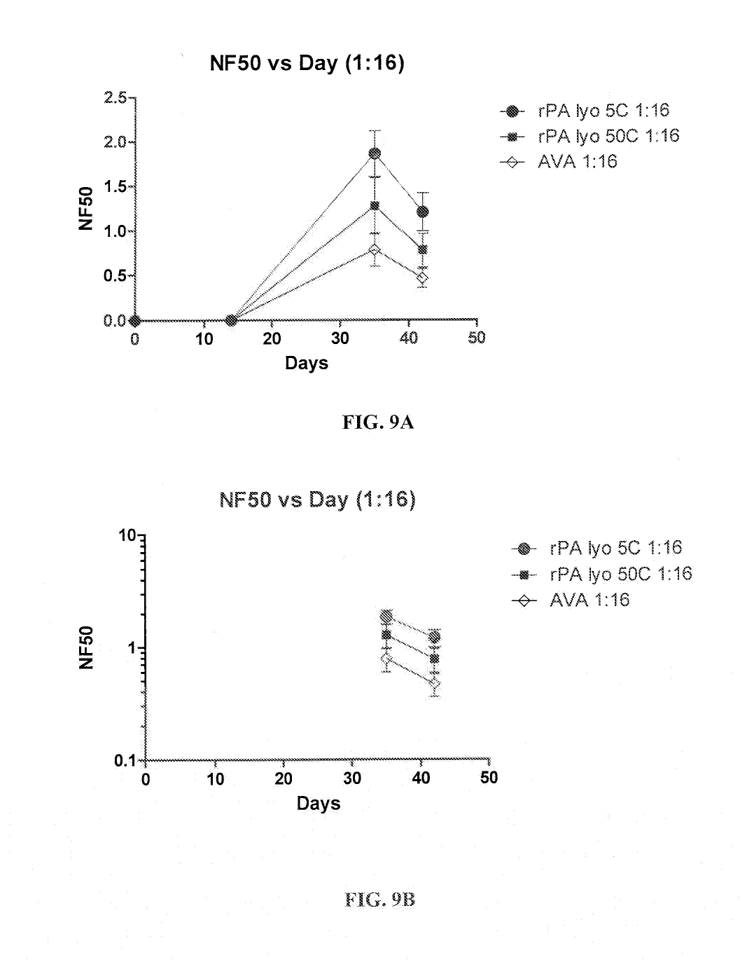

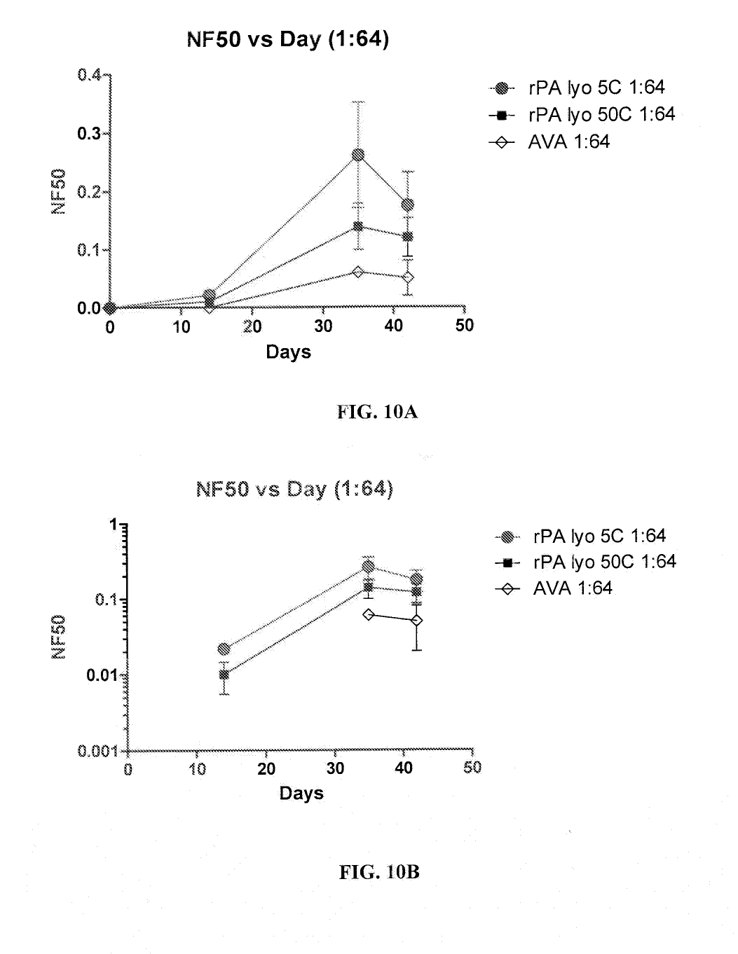

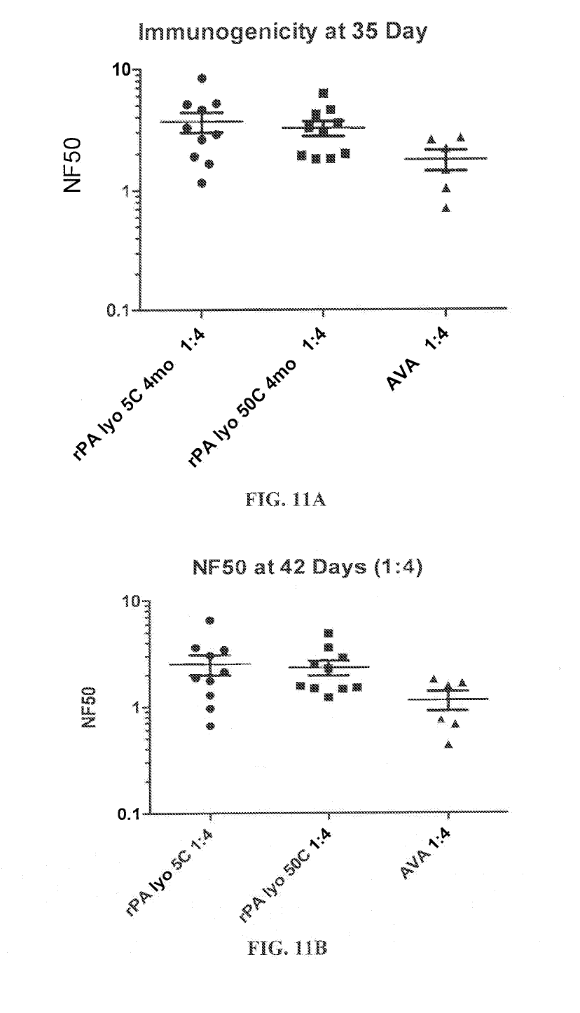

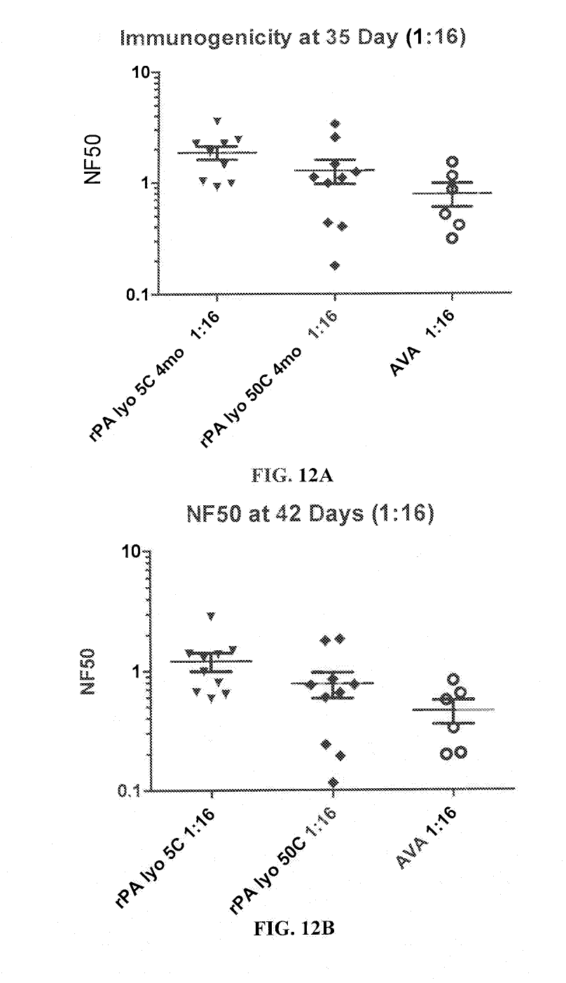

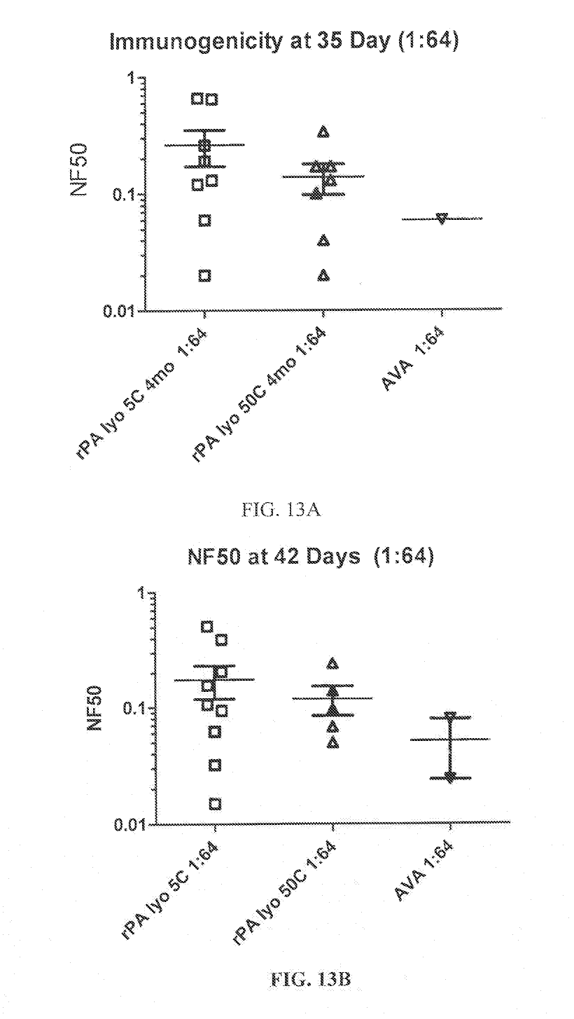

All animals tested negative for toxin neutralizing activity at day 0. <NF50> reached maximum levels at 35 days for all three tested vaccines at all three dose levels (FIGS. 8A-B, 9A-B, and 10A-B), showing that rPA had similar or better immunogenicity kinetic as AVA at all three doses. As shown in FIG. 8A-B, lyophilized rPA (1:4 dilution) stored at both 5 and 50° C. had a higher <NF50> than AVA (1:4 dilution). Similarly, at dilutions of 1:16 and 1:64, the lyophilized rPA had higher <NF50> than the comparable AVA dilution (see FIGS. 9A-B and 10A-B).

For the 1:4 dilutions at 35 days, the <NF50>geomean (gin) for lyophilized rPA stored at 5 and 50° C. was found to be 3.14 and 2.98, respectively; and the <NF50>gm of AVA at 1:4 dilution was 1.61. There was no statistical differences in <NF50>gm between the lyophilized rPA formulations (1:4) stored at 5° C. versus 50° C., p=0.82 (t. test). The <NF50>gm of the combined data (rPA lyo 5 and 50° C.) was found to be 3.06; and the combined <NF50>gm was statistical higher than that of the AVA reference (1.61), p=0.034 (t. test). At day 42 (1:4), there was no statistical different in <NF50>gm between rPA lyo stored at 5 versus 50° C. (2.07 and 2.13, respectively), p=0.92 (t. test); and the combined <NF50>gm was found to be 2.10. The combined <NF50>gm of 2.10 was statistically higher than that of AVA reference (1.01), p=0.028 (t. test).

For the 1:16 dilutions at day 35, the <NF50>gm for lyophilized rPA stored at 5 and 50° C. was found to be 1.70 and 0.94, respectively. There was no statistical differences in <NF50>gm between rPA lyo stored at 5 and 50° C., p=0.081 (t. test). The combined <NF50>gm was found to be 1.27, and there was no statistical difference in combined <NF50>gm for rPA lyo (5 and 50° C.) and that of AVA reference (0.67), p=0.064 (t. test). At day 42 (1:16), there was no statistical different in <NF50>gm between rPA lyo stored at 5 versus 50° C. (1.08 and 0.57, respectively), p=0.071 (t. test); and the combined <NF50>gm was found to be 0.78. The combined <NF50>gm of 0.78 was statistical higher than that of AVA reference (0.40), p=0.05 (t. test).

For the 1:64 dilutions at day 35, the <NF50>gm for lyophilized rPA stored at 5 and 50° C. were not statistically different (1.06 and 0.10, respectively), p=0.386 (t. test). The combined <NF50>gm was found to be 0.13 and the AVA reference was 0.06. At day 42 (1:64), there was no statistical different in <NF50>gm between rPA lyo stored at 5 and 50° C. (0.11 and 0.10, respectively), p=0.91 (t. test); and the combined <NF50>gm was found to be 0.11. There was no statistical difference between the combined <NF50>gm of 0.11 and the AVA reference (0.04), p=0.35 (t. test).

Geometric mean of NF50 (<NF50>gm) of lyophilized rPA stored at 5 and 50° C. compared to AVA at three dilutions (1:4, 1:16, and 1:64) at days 35 and 42 are shown in FIGS. 11A-B, 12A-B, and 13A-B.

At day 35, the <NF50>gm for the lyophilized rPA vaccine stored at 50° C. was found to be 2.98, 0.94 and 0.1 for doses 1:4, 1:16 and 1:64, respectively. The <NF50>gm of the lyophilized rPA vaccine stored at 5° C. were similar at day 35 (3.14, 1.7 and 0.16, respectively) with no statistically significant difference compared to 50° C. (alph=0.05). Similar results were found at day 42. These data demonstrated that the immunogenicity of the lyophilized rPA vaccine stored at 50° C. for 4 months was not significantly different from the lyophilized rPA vaccine stored at 5° C.

At day 35, the combined <NF50>gm (5° C. and 50° C.) was found to be 3.06, 1.27 and 0.1 at doses 1:4, 1:16 and 1:64, respectively; and the p values for the combined <NF50>gm compared to AVA at 1:4 and 1:16 were p=0.034 and p=0.064, respectively. The combined <NF50>gm was found to be non-inferior (statistical higher or no difference) to that of the AVA. Similar results are shown for day 42. At 42 days, the combined <NF50> values for 1:4, 1:16, and 1:64 were 2.10, 0.78, 0.11, respectively; and the p values for the combined <NF50>gm compared to AVA at 1:4, 1:16, and 1:64 were p=0.92, p=0.071, and p=0.91, respectively. These immunogenicity data show that the rPA lyophilized vaccine stored at 5° C. and 50° C. for 4 months was at least as immunogenic as the AVA vaccine.

In sum, these results show that the tested lyophilized formulation was capable of stabilizing the rPA vaccine for at least 4 months at 50° C. The data demonstrated that the rPA lyophilized formulation had superior thermal stability profile compared to AVA vaccine. Thus, the results showed that the rPA lyophilized formulation was effective for rPA anthrax vaccine storage, and the tested formulation was room temperature stable and able to circumvent a cold chain distribution.

### Example 5

### Guinea Pig Immunogenicity Study

A guinea pig immunogenicity study is outlined in Table 8.

TABLE 8
Guinea Pig Immunogenicity Study Design

| | Pre- | | | |
|---|---|---|---|---|
| | dilu- | Dilu- | GP Required(n) | Reported |
| Vaccine | tion | tion | 315 to 385 g | Results |
| AVA | None | 1/1.6 | 6 male + 6 female (12) | Survivors/Total |
| Reference | | ¼ | 6 male + 6 female (12) | Survivors/Total |
| Lot | | 1/10 | 6 male + 6 female (12) | Survivors/Total |
| | | 1/25 | 6 male + 6 female (12) | Survivors/Total |
| rPA102 | None | 1/1.6 | 6 male + 6 female (12) | Survivors/Total |
| Fresh | | ¼ | 6 male + 6 female (12) | Survivors/Total |
| | | 1/10 | 6 male + 6 female (12) | Survivors/Total |
| | | 1/25 | 6 male + 6 female (12) | Survivors/Total |
| rPA102 | None | 1/1.6 | 6 male + 6 female (12) | Survivors/Total |
| Liquid | | ¼ | 6 male + 6 female (12) | Survivors/Total |
| 12-15 months | | 1/10 | 6 male + 6 female (12) | Survivors/Total |
| at 5° C. | | 1/25 | 6 male + 6 female (12) | Survivors/Total |
| rPA102 | None | 1/1.6 | 6 male + 6 female (12) | Survivors/Total |
| Lyophilized | | ¼ | 6 male + 6 female (12) | Survivors/Total |
| 1 month at | | 1/10 | 6 male + 6 female (12) | Survivors/Total |
| 40° C. (then | | 1/25 | 6 male + 6 female (12) | Survivors/Total |
| reconstituted) | | | | |

| Challenge Preparation | | | |
|---|---|---|---|
| Colony Forming | 40 | 4 male + 4 female (8) | Deaths/Total |
| Units per | | | |
| 0.1 mL dose | | | |

| | |
|---|---|
| Total number of | 200 (100 males and |
| vaccinated animals | 100 females) |

### Example 6

### Immunogenicity and Physiochemical Stability in Mice

Three lyophilized rPA vaccine formulations were tested for immunogenicity and physiochemical stability compared to liquid rPA vaccine. The content and purity of rPA vaccines (physiochemical stability properties) were measured by macrophage lysis assay (MLA), size exclusion chromatography (SEC-HPLC), and anion exchange chromatography (AEX-HPLC). The immunogenicity of the rPA vaccines was evaluated by vaccinating CD-1 mice with four dose levels and testing the ability of the mice serum to neutralize anthrax toxin (NF50).

Liquid rPA Vaccine Formulation

Liquid rPA formulation (F1) was prepared under aseptic condition at 0.15 mg/mL rPA, 1.5 mg/mL alum, 2% Alanine, 0.01% TWEEN 80, 25 mM NaPi, pH 7.0. The samples for the stability assays contained 5 mL liquid suspension filled in 10 mL glass vial (10 doses per vial). The intended human dose is 75 μg rPA/750 μg alum per 0.5 mL with intramuscular injection.

Lyophilization rPA Vaccine Formulations

Three lyophilized rPA formulations were prepared. The final formulations, prior to lyophilization, are shown in Table 9. The first lot (lyoA) was formulated with 0.15 mg/mL rPA, 1.5 mg/mL alum, 20% Trehalose, 2% Ala, 0.025% Tw80 and 5 mM NaPi at pH 7.0. The second and third lots (lyoB and lyoC, respectively) contained 3.3× higher rPA and alum concentrations (0.5 mg/mL and 5.0 mg/mL, respectively) with slight variations in sugar, amino acid and buffer as indicated in Table 9.

TABLE 9
Concentration of Final Formulation Blended Prior to Lyophilization

| | rPA | Alum | | Amino | TWEEN | | Liq-Lyo |
|---|---|---|---|---|---|---|---|
| Lot # | (mg/mL) | (mg/mL) | Sugar | Acid | 80 | Buffer | Vol, mL |
| LyoA | 0.15 | 1.5 | 20% | 2% | 0.025% | 5 mM Napi, | 2 mL fill |
| | | | Trehalose | Alanine | | pH 7.0 | in 10 mL |
| | | | | | | | vials |
| LyoB | 0.5 | 5 | 20% | 2% | 0.025% | 5 mM Napi, | 2 mL fill |
| | | | Trehalose | Alanine | | pH 7.0 | in 10 mL |
| | | | | | | | vials |
| LyoC | 0.5 | 5 | 20% | 2% | 0.025% | 20 mM Tris, | 2 mL fill |
| | | | Trehalose + | Glycine | | pH 7.4 | in 10 mL |
| | | | 1% | | | | vials |
| | | | Sucrose | | | | |

All three rPA lyophilized formulations were designed such that most rPA protein was bound to alum with little or no free rPA protein in the solution. 2 mL liquid suspension was filled into a 10 mL glass vial. Lyophilization was performed using FTS LyoStar® II with the following processing parameters.

### Initial Freezing:

| Step | Process | Setting |
|---|---|---|
| Freeze | Freeze (° C.) | −60 |
| | Additional Freeze time (min) | 0 |
| | Condenser (° C.) | −80 |
| | Vacuum (mTorr) | 90 |

### Drying:

| | Temp | Time | | Vac |
|---|---|---|---|---|
| Step | ° C. | (min) | R/H | mTorr |
| 1 | −60 | 120 | H | 90 |
| 2 | −28 | 60 | R | |
| 3 | −28 | 1250 | H | |
| 4 | −28 | 550 | H | |
| 5 | 25 | 480 | R | |
| 6 | 25 | 600 | H | |
| 7 | 30 | 120 | R | |
| 8 | 30 | 300 | H | |
| 9 | 35 | 120 | R | |
| 10 | 35 | 300 | H | |
| 11 | 40 | 120 | R | |
| 12 | 40 | 300 | H | |
| 13 | 45 | 120 | R | |
| 14 | 45 | 255 | H | |

### Post-Drying:

| | |
|---|---|
| Secondary Dry Set-Point Temperature | +65° C. |

Post-Heat Settings

| | |
|---|---|
| Temperature (° C.) | +25 |
| Time (min) | 1250 |
| Vacuum (mTorr) | 1250 |

Prior to vaccination and testing, the lyophilized samples were reconstituted with water for injection (WFI) to produce a final concentration of 0.15 mg/mL rPA and 1.5 mg/mL alum for all three formulations. The final concentration was accomplished by adding 1.55, 6.2 and 6.18 mL of WFI to vials of the first lot (LyoA), second lot (LyoB) and third lot (LyoC), respectively. The final suspension volume per vial for LyoA was 2.0 mL and for both lots LyoB and LyoC were 6.7 mL. The concentration of rPA vaccines after reconstitution are shown in Table 10.

TABLE 10
Concentration of lyophilized rPA vaccine after reconstitution

| | | | | | | | | Recons | Final | # Dose/vial |
|---|---|---|---|---|---|---|---|---|---|---|
| | rPA | Alum | | Amino | TWEEN | | | WFI Vol, | Vol, | (0.5 mL/ |
| Lot # | (mg/mL) | (mg/mL) | Sugar | Acid | 80 | NaCl | Buffer | mL | mL | dose) |
| Lyo | 0.15 | 1.5 | 20% | 2% | 0.025% | — | 5 mM | 1.55 | 2.0 | 4.0 |
| A | | | Trehalose | Alanine | | | Napi, | | | |
| | | | | | | | pH 7.0 | | | |
| Lyo | 0.15 | 1.5 | 6% | 0.6% | 0.075% | — | 1.5 mM | 6.2 | 6.7 | 13.3 |
| B | | | Trehalose | Alanine | | | Napi, | | | |
| | | | | | | | pH 7.0 | | | |
| Lyo | 0.15 | 1.5 | 6% | 0.6% | 0.075% | — | 6 mM | 6.18 | 6.7 | 13.3 |
| C | | | Trehalose + | Glycine | | | Tris, | | | |
| | | | 0.3% | | | | pH 7.4 | | | |
| | | | Sucrose | | | | | | | |

The total number of doses per vial in lots LyoB and LyoC were higher than that of lot LyoA (13.3 vs 4.0). The manufacturing cost of a higher doses vial (such as 13.3 doses per vial) is significantly lower than that of a lower dose vial (e.g., 4 doses per vial). Thus, it was an economical preference to develop a formulation and process for producing higher dose vials.

### Stability & Test Assays

The rPA liquid formulation (F1) was placed in a long term stability program at storage temperatures of 5, 25 and 40° C. The three rPA lyophilized lots (LyoA, LyoB, and LyoC) were placed in a long term stability program at storage temperatures of 5, 25, 40, and 50° C. The physiochemical tests were performed for both the liquid (F1) and lyophilized rPA vaccine formulations. Four (4) months of stability data for the samples were collected.

The physiochemical properties of the rPA test vaccines were evaluated by a series of assays, i.e., MLA, SEC-HPLC and AEX-HPLC. All of these assays were performed using rPA proteins extracted from the alum. The extraction procedure utilized 200 mM potassium phosphate/0.01% Tw80/0.9% NaCl.

A rPA bulk drug substance (BDS) stored at −80° C. was used as a reference control. The BDS control was purified from a ΔSterne-1(pPA102)CR4 strain of B. anthracis, which was developed by the U.S. Army Medical Research Institute of Infectious Diseases (USAMRIID) as an asporogenic, non-toxigenic expression system for the production of rPA. The rPA BDS was purified and stored in 20 mM Tris, 0.9% NaCl, pH 7 under −80° C. frozen condition.

I. Macrophage Lysis Assay (MLA)

The in vitro macrophage lysis assay (MLA) was used to determine the cytotoxicity of rPA on the murine macrophage cell line J7774A.1. The MLA measures the activity of rPA and rLF toxin. The assay involves adding rPA protein to lethal factor protein (rLF) to form a lethal toxin complex, which caused pore formation with the cell membranes of the macrophages, leading to cell lysis.

The activity of the rPA lyophilized vaccines (using rPA desorbed from alum) was measured relative to a BDS reference standard and reported as a percentage of the reference standard. The percentage of cells surviving toxin challenge was determined. For example, 100% MLA activity of rPA vaccine would indicate there was no loss in the cytotoxicity activity of rPA adsorbed to alum and after lyophilized. In brief, microphage cells were seeded in a 96 well plate at 5×10⁴ cells/well and the plate was placed in a CO₂ incubator overnight. The next day, 100 ul of serial diluted rPA test samples or rPA reference standard (starting rPA concentration was 800 ng/ml and then 1:2 diluted down to 0.8 ng/mL) was mixed with rLF (the rLF concentration was constant at 100 ng/ml) and added to the wells. Four hours later, 25 ul of MTT (3-(4,5-Dimethylthiazol-2-yl)-2,5-diphenyltetrazolium bromide) at 5 mg/mL was added to each well and the plate was incubated for 1.5 hours. After the 1.5 hour incubation, 100 ul of soluble solution (20% SDS in 50% dimethylformamide solution) was added to the wells and the plate was incubated at 37° C. overnight. The next day, the plate was read by a plate reader at 570 nm and OD readings were graphed with 4-parameter model using software (SoftMax). The ED50 value of the test sample rPA was then compared to that of the rPA reference standard, and their ratio was used to report relative activity of the rPA test sample. The accuracy of the assay was determined to be +/−30%.

II. Size-Exclusion Chromatography (SEC-HPLC)

SEC-HPLC is a chromatographic technique used to separate proteins based on their molecular weight and size and it is commonly used to assess the stability of a protein in a formulation. A larger protein typically has a shorter retention time (elute out sooner) in a SEC-column than a smaller protein. Protein that is degraded (or fragmented) will become smaller in size and elute out of the chromatography later. Vice versa, aggregated protein will elute sooner. For example, an aggregated rPA protein that has increased in size would have a shorter retention time than the native rPA protein, and a degraded rPA protein (breakdown in size) would have a longer retention time.

SEC-HPLC is a common assay used to assess the physiochemical stability of a protein in a formulation. Compared to MLA, the SEC-HPLC is, in general, relatively more sensitive and quantitative with precision of less than 5% in % peak area and less than 0.1% in retention time within the same run.

The SEC-HPLC was performed using a TSK G3000SWXL column (Tosoh BioScience P/N M1182-05M) with a mobile phase of 50 mM sodium phosphate/250 mM potassium chloride at pH 7.4. An ultraviolet (UV) detector at 215 nm or fluorescence detector at 280 nm (excitation) and 335 nm (emission) were used for the detection.

III. Anion Exchange Chromatography (AEX-HPLC)

AEX-HPLC separates proteins based on their net electrostatic charge. In general, the assay involved injecting rPA protein solution into an HPLC equipped with an anion exchange column. The rPA protein was retained by the anion exchange (AEX) column (stationary phase) due to electrostatic interaction. The rPA protein was then eluted out by using mobile phase with gradually increased ionic strength (NaCl concentration). The ionic strength (or elution time) at which the rPA molecules eluted is related to its net charges.

The rPA molecule is known to be susceptible to degradation by deamidation mechanism (D'Souza, Journal of Pharmaceutical Sciences, 102(2):454-461, 2013) resulting in a change in net change. Deamidation increases the negative charge of the rPA protein due to the conversion of asparagine residues to aspartate (more negative). The deamidated rPA (typically referred to as the acidic species) elutes in higher ionic strength solution and has a longer elution time than the native rPA protein. The purity of the rPA was characterized by the % main peak.

The AEX-HPLC was performed using Hamilton PRP-X500 Anion Exchange Column (P/N 79641) using mobile phase A of 25 mM Tris at pH 8.0 and mobile phase B of 25 mM Tris, 0.5 M NaCl at pH 8.0. Similar to SEC-HPLC, UV or fluorescence detectors were used.

IV. Immunogenicity Test

Immunogenicity was evaluated using toxin neutralizing assay (TNA) (Hering et al., Biologicals 32 (2004) 17-27; Omland et al., Clinical and Vaccine Immunology (2008) 946-953; and Li et al., Journal of Immunological Methods (2008) 333:89-106. ED50 values for the mouse reference serum (prepared as described above) were used to determine the NF50 values of the test serum sample and the positive control. The first month stability data are reported herein.

For the in vivo immunogenicity test, 20 female CD-1 mice per group received a single 0.5 mL intraperitoneal (i.p.) injection of LyoA, LyoB, LyoC, or liquid rPA (F1). Four dilution dose levels were evaluated (1:4, 1:8, 1:16 and 1:32). The dilutions were prepared on the day of the vaccination with saline solution. Serum samples were obtained by cardiac bleeding at Day 28 after vaccination.

Physiochemical Stability Results

Table 11 summarizes the MLA, SEC-HPLC & AEX-HPLC results of the three lyophilized formulations stored at 4 months at 5, 25, 40 and 50° C. and the BDS reference control.

TABLE 11
Summary of physiochemical stability data at 4 months
4 months physiochemical stability data

| | | MLA % | SEC % | AEX % |
|---|---|---|---|---|
| | Temp, | Relative to | Purity, | Purity, |
| Name | ° C. | Reference | Main Peak | Main Peak |
| Ref BDS | −80 | 100 | 84.0 | 80.8 |
| lyoA | 5 | 116 | 85.8 | 87.1 |
| | 25 | 102 | 85.5 | 86.6 |
| | 40 | 107 | 83.1 | 84.7 |
| lyoB | 5 | 111 | 84.9 | 87.8 |
| | 25 | 112 | 84.9 | 86.2 |
| | 50 | 108 | 84.5 | 85.3 |
| lyoC | 5 | 117 | 84.7 | 87.5 |
| | 25 | 123 | 84.8 | 84.5 |
| | 50 | 104 | 84.7 | 70.5 |
| Ref BDS | −80 | 100 | 98.6 | 78.2 |
| Liq. rPA | 5 | 98 | 95.3 | 65.5 |
| F1, | 25 | 65 | 91.4 | 25.6 |
| 1 month | 40 | 0 | 0 | 0 |

Macrophage Lysis Assay Results

These MLA results show that there was no significant drop in rPA cytotoxicity for the three lyophilization formulations stored at 4 months at temperatures up to 40 and 50° C. when compared to the BDS reference control (within error variability of +/−30%).

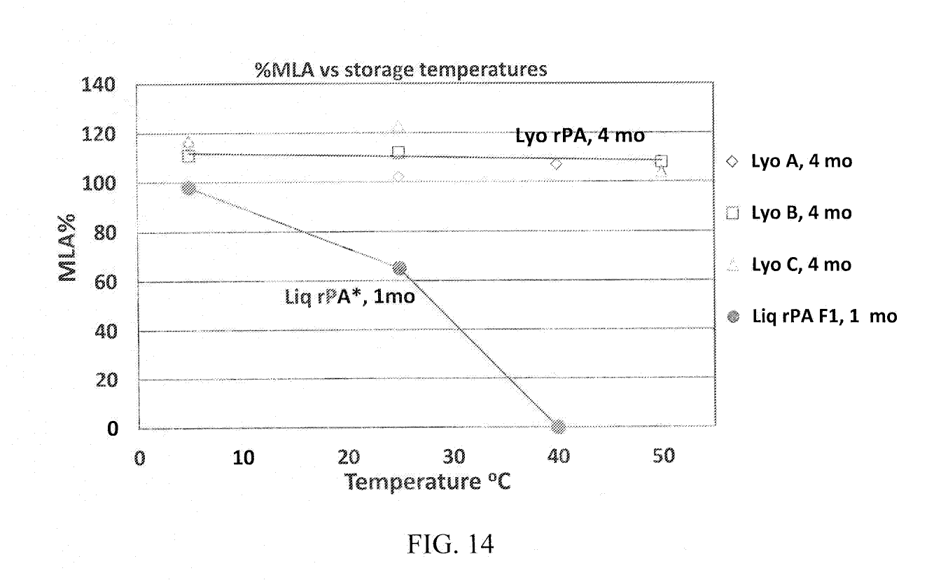

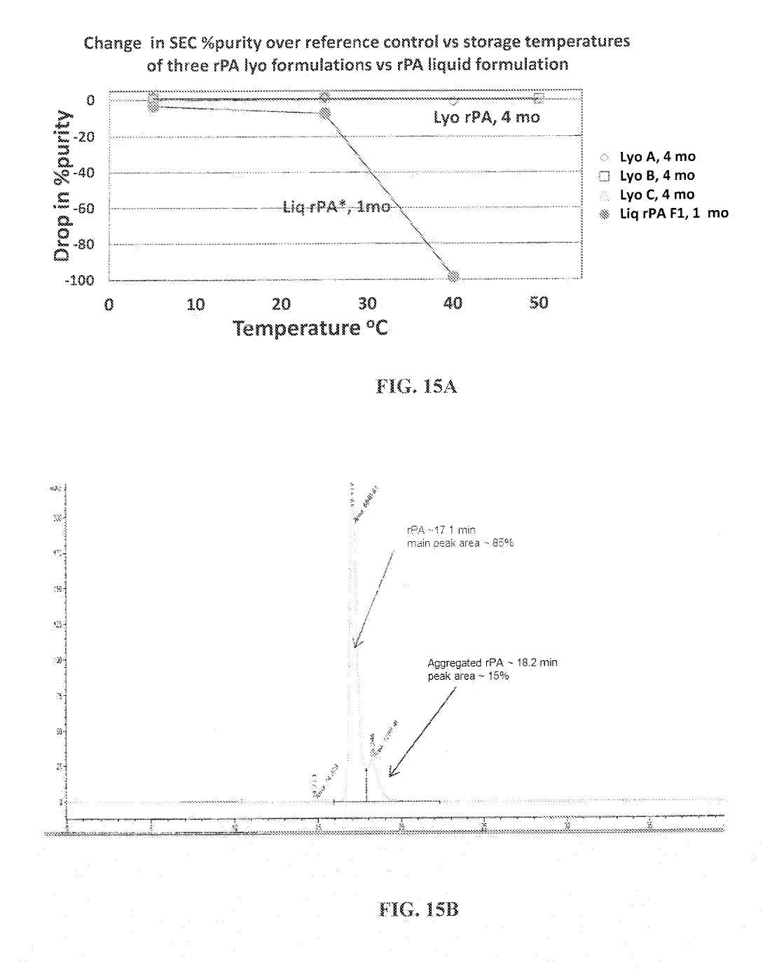

In comparison, there is a significant drop in MLA value for the liquid rPA vaccine (F1) stored for one month at 5, 25 and 40° C. The MLA values were found to be 98%, 65% and 0% at 5, 25 and 40° C., respectively. FIG. 14 shows a comparison of the MLA % as a function of storage temperature for the three lyophilized lots stored for 4 months versus the rPA liquid lot (F1) stored for 1 month. These results show that the three lyophilized formulations maintained the MLA activity of the rPA vaccine for at least up to 4 months at 40 and 50° C., whereas the liquid rPA vaccine loss all it MLA activity after storage for 1 month at 40° C.

Size-Exclusion Chromatography (SEC-HPLC) Results

A typical SEC chromatograph of the rPA reference control (BDS) is shown in FIG. 15B. The percent peak area was calculated for each sample peak. The native rPA protein (monomer) eluted at 17.1 minutes with a % main peak area of 85%. The second peak eluted at 18.2 minutes and corresponds to the aggregated rPA molecule main peak area of ˜15%. The % purity of rPA protein was determined by the % area of the 17.1 minutes elution peak. i.e., the purity of the rPA protein (% purity rPA)=the percent peak area of the rPA peak (corresponding to monomer rPA with retention time of ˜17.1 min)/the total peak area).

The purity results for the LyoA, LyoB and LyoC were comparable to the purity results for the rPA reference BDS of 84.0% stored at −80° C., see Table 11. The SEC-HPLC data showed that there was no significant drop in purity compared to the reference control for all three lyophilized formulations stored at 4 months at all storage temperatures.

As a comparison, there was a significant drop in purity for the liquid rPA vaccine when stored at an accelerated temperature (25, 40, or 50° C.) for 1 month. The relative % purity of rPA of the liquid formulation dropped −3.3%, −7.2% and −98.6% at 5, 25 and 40° C., respectively.

The % purity of the BDS reference control were found to be 84.0% and 98.6% for the lyophilized and the liquid assays, respectively. The two tests were performed at different times. The difference in % purity of the reference control was not unusual. The difference could be due to varying SEC-column condition and sample preparation procedure. The relative % purity (over reference control) is typically used to compare the stability samples at different times and across different laboratories.

FIG. 15A shows the relative decrease of rPA SEC % purity (over BDS reference control) as a function of storage temperature for the three lyophilized formulation compared to the liquid formulation. The data demonstrated there was no change in SEC-purity for the lyophilized rPA vaccine stored for at least 4 months at 40 or 50° C., whereas the liquid rPA vaccine was completely degraded after 1 month storage at 40° C.

Anion Exchange Chromatography (AEX-HPLC) Results

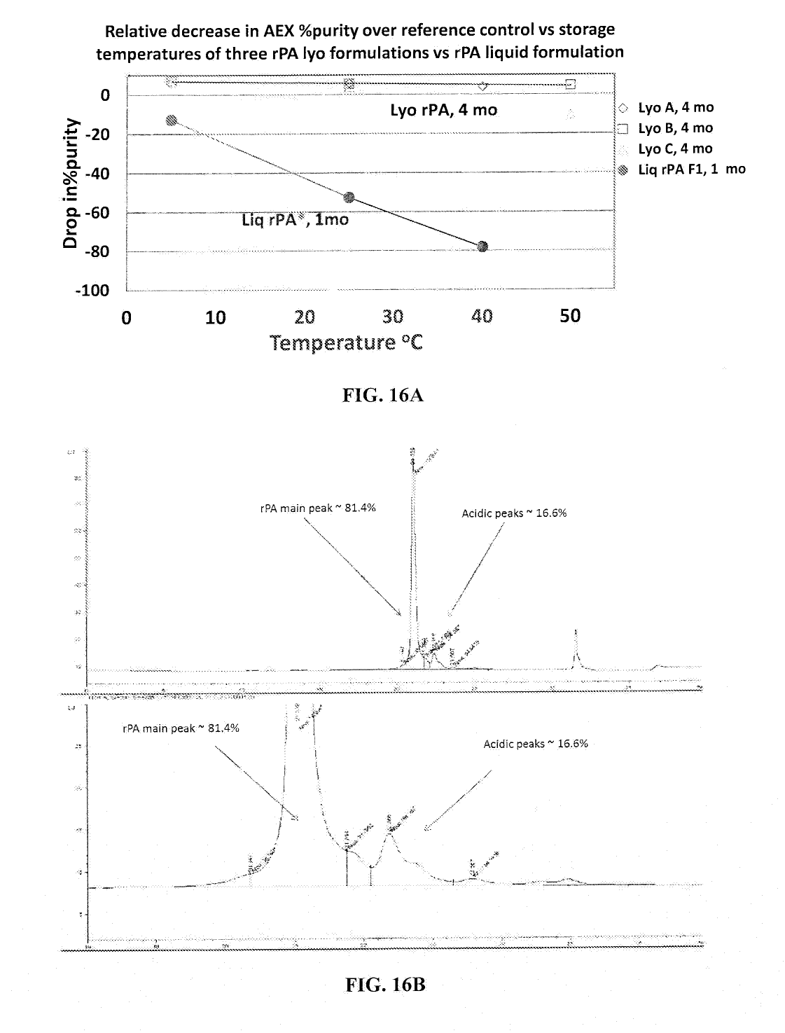

A typical AEX chromatograph for the rPA reference control (BDS) is shown in FIG. 16B. The purity of rPA was characterized by the % main peak area. The native rPA protein eluted at 21.0 minutes with a main peak of ˜81.4%. The deamidated rPA (typically referred to acidic species) eluted between 21.7 and 22.4 minutes with an area of 16.6%. The % main peak area was calculated by first integrating the area under the curve of all peaks (except the buffer peak) and then the % main peak area corresponds to the rPA peak with ˜retention time of 21.0 min, (i.e., % main peak area=peak area (RT=21.0 min)/Sum of all peak areas). Similar to SEC-HPLC, the purity of rPA was the same as % main peak area. The accuracy of the AEX assay was about 10-15%.

The AEX % purity of the first lyophilized lot (lyoA) stored for 4 months was found to be 87.1, 86.6, 84.7 at 5, 25 and 40° C., respectively. The AEX purity for the second lyophilized lot (lyoB) was 87.8%, 86.2% and 85.3% at 5, 25 and 50° C., respectively. The AEX % purity for the third lyophilized lot (lyoC) was 87.5%, 84.5% and 70.5% at 5, 25 and 50° C., respectively. These purity results for LyoA, LyoB, and LyoC were comparable to the purity of the rPA reference control (BDS) of 80.8%. There is no significant drop in purities over the reference control for all three lyophilized formulations after 4 months storage at all storage temperatures.

The AEX purity of the liquid rPA vaccine stored at 1 month was found to be 65.5%, 25.6% and 0% at 5, 25 and 40° C., respectively; and the AEX purity for rPA reference control (BDS) was 78.2% when used in the assay testing liquid rPA. The AEX purity results are summarized in Table 11.

As shown in FIG. 16A, there was a significant drop in the AEX-purity in the rPA liquid formulation (F1) at 5, 25 & 40° C. compared to the lyophilized formulations. The AEX-data demonstrated that there was no loss in purity of the lyophilized rPA vaccine stored for at least 4 months at 40 or 50° C.; whereas, the liquid vaccine was completely degraded at 1 month after storage at 40° C.

In Vivo Immunogenicity Results

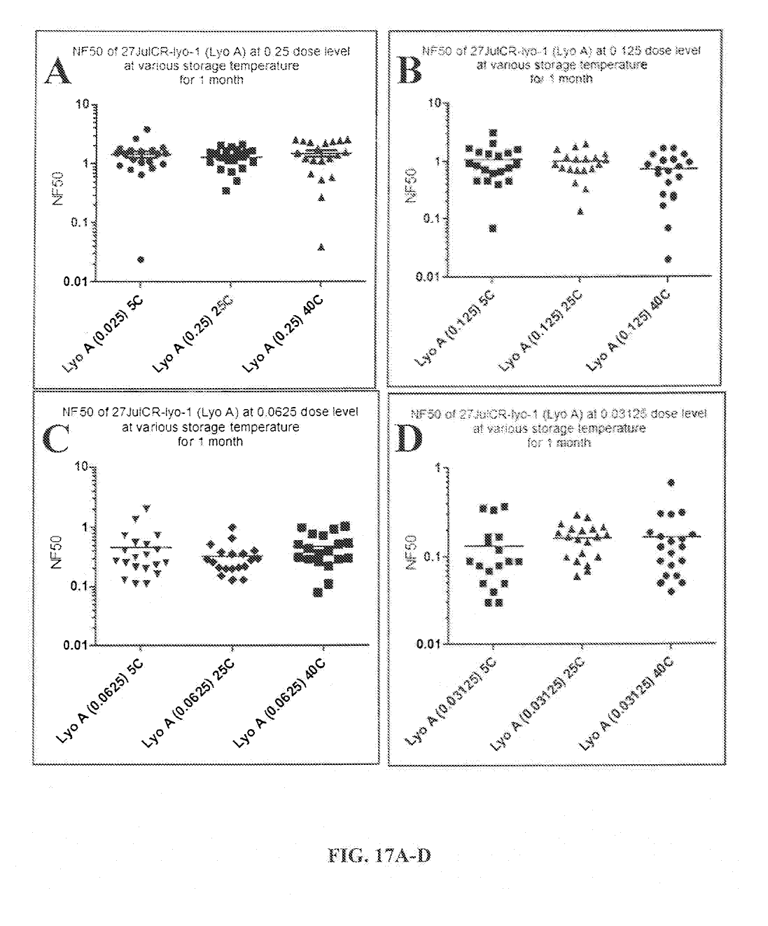

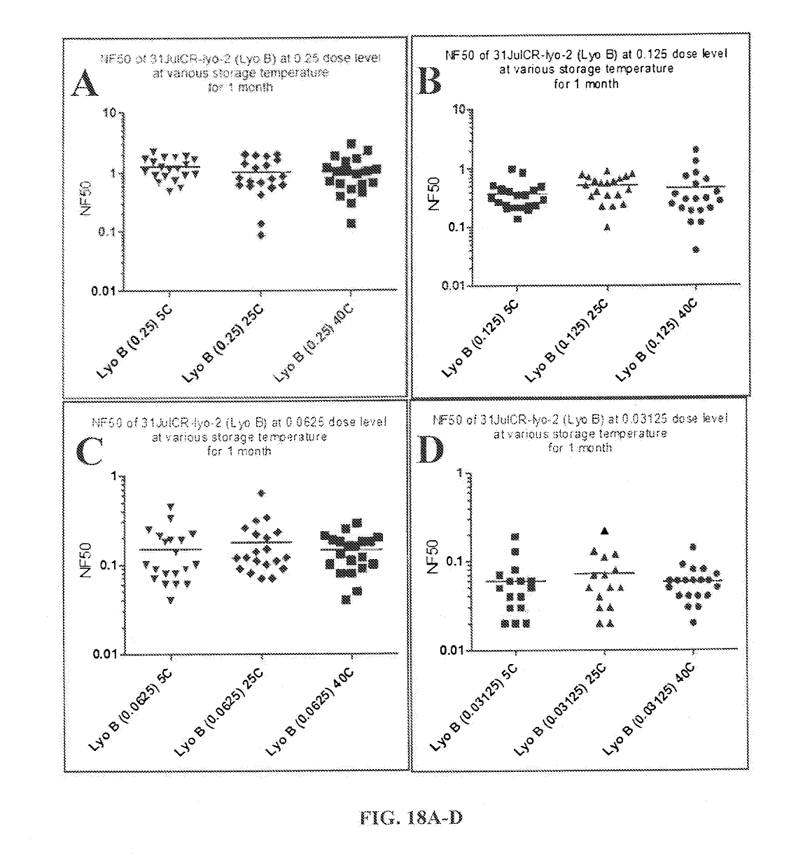

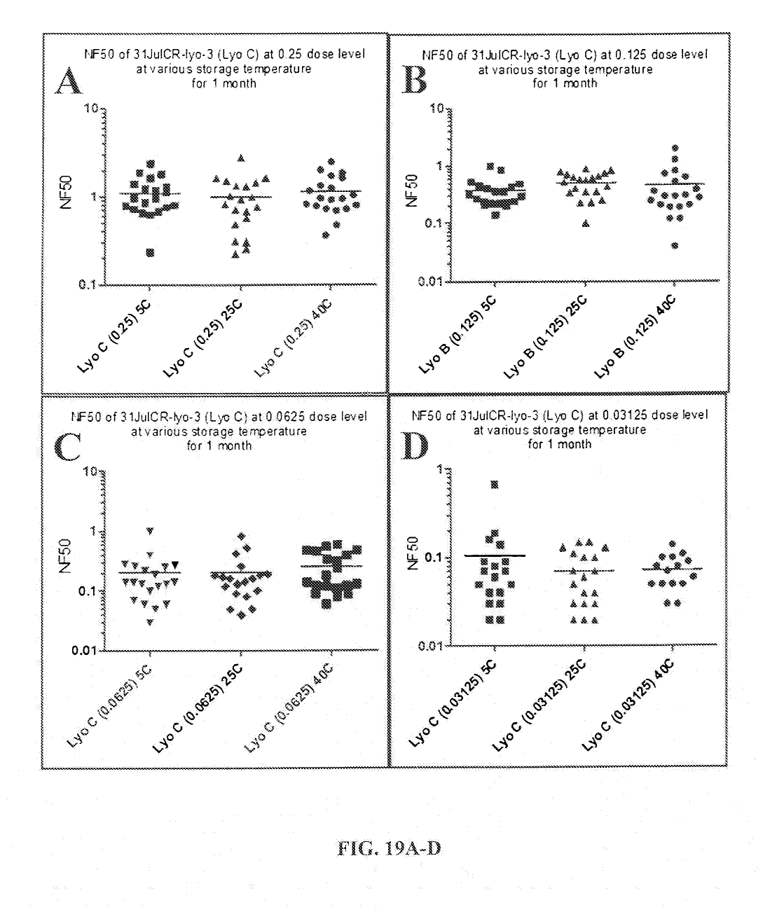

The NF50 results in mice at dose levels 0.25, 0.125, 0.0625 and 0.03125 for the three lyophilized formulations stored for 1 month each were determined. FIGS. 17A-D, 18A-D, and 19A-D compares the NF50 data (n=20) across various temperatures (5, 25 and 40° C.) at their corresponding formulation and dose level.

The geometric mean of the NF50 (<NF50>gm) at 5, 25 and 40° C. for the four doses level of the three lyophilization formulations are summarized in Tables 12A-C.

Tables 12A-C. GeoMean of NF50 (n=20) for the three lyophilized formulations at 5, 25 & 40° C. at four dose levels (0.25, 0.125, 0.0625 & 0.03125)

13A. <NF50> gm of Lyo A stored at 5, 25 and
40° C. at one month at four dose levels

| | | | | Are the <NF> gm |
|---|---|---|---|---|
| | | | | significantly different |
| | | | | among temperatures? |
| Dose Level | 5° C. | 25° C. | 40° C. | (p < 0.050) ANOVA Test |
| 0.25 | 1.12 | 1.20 | 1.13 | No, p = 0.97 |
| 0.125 | 0.84 | 0.86 | 0.50 | No, p = 0.10 |
| 0.0625 | 0.33 | 0.28 | 0.39 | No, p = 0.36 |
| 0.03125 | 0.10 | 0.15 | 0.13 | No, p = 0.18 |

13B. <NF50> gm of Lyo B stored at 5, 25, 40 and
50° C. at one month at four dose levels

| | | | | Are the <NF> gm |
|---|---|---|---|---|
| | | | | significantly different |
| | | | | among temperatures? |
| Dose Level | 5° C. | 25° C. | 40° C. | (p < 0.050) ANOVA Test |
| 0.25 | 1.16 | 0.81 | 0.86 | No, p = 0.22 |
| 0.125 | 0.33 | 0.46 | 0.32 | No, p = 0.20 |
| 0.0625 | 0.12 | 0.15 | 0.13 | No, p = 0.59 |
| 0.03125 | 0.05 | 0.06 | 0.05 | No, p = 0.78 |

13C. <NF50> gm of 31 Lyo C stored at 5, 25, 40
and 50° C. at one month at four dose levels

| | | | | Are the <NF> gm |
|---|---|---|---|---|
| | | | | significantly different |
| | | | | among temperatures? |
| Dose Level | 5° C. | 25° C. | 40° C. | (p < 0.050) ANOVA Test |
| 0.25 | 0.98 | 0.80 | 1.02 | No, p = 0.37 |
| 0.125 | 0.43 | 0.45 | 0.46 | No, p = 0.94 |
| 0.0625 | 0.13 | 0.15 | 0.20 | No, p = 0.21 |
| 0.03125 | 0.07 | 0.06 | 0.07 | No, p = 0.78 |

For lot LyoA after storage for 1 month, the <NF50>gm were found to be 1.12, 1.20, and 1.13 at 5, 25 and 40° C. respectively at dose level 0.25. There was no statistically significant difference in the <NF50>gm among the three storage temperatures (5, 25 and 40° C.) at p=0.97. Similarly, it was also shown that there was no statistical difference in <NF50>gm among the various storage temperatures at the other tested dose levels (0.125, 0.0625 and 0.01315) for all three lyophilized formulations (LyoA, LyoB, and LyoC). The NF50 data demonstrated that there was no significant drop in immunogenicity for the three lyophilized formulations at 1 month up to 40° C.

In contrast, the liquid rPA formulation (F1) showed a significant drop in immunogenicity at the 25 and 40° C. storage temperatures at 1 month. Table 13 shows the <NF50>gm results for the rPA liquid formulation stored at 1 month at 5, 25 and 40° C.

TABLE 13
Geomean of NF50 of liquid rPA formulation F1 stored at 1 month
at 5, 25 & 40° C. at four dose levels (0.3, 0.2, 0.1 & 0.05)
<NF50> gm of Liquid rPA (F1) formulation stored at 5,
25 and 40° C. at one month at four dose levels

| | | | | Are the <NF> gm |
|---|---|---|---|---|
| | | | | significantly different |
| | | | | among temperatures? |
| Dose Level | 5° C. | 25° C. | 40° C. | (p < 0.050) ANOVA Test |
| 0.3 | 1.26 | 0.69 | 0.30 | Yes, p = 0.0023 |
| 0.2 | 1.18 | 0.55 | 0.13 | Yes, p = <0.0001 |
| 0.1 | 1.03 | 0.22 | 0.09 | Yes, p = <0.0001 |
| 0.05 | 0.32 | 0.15 | 0.03 | Yes, p = 0.0010 |

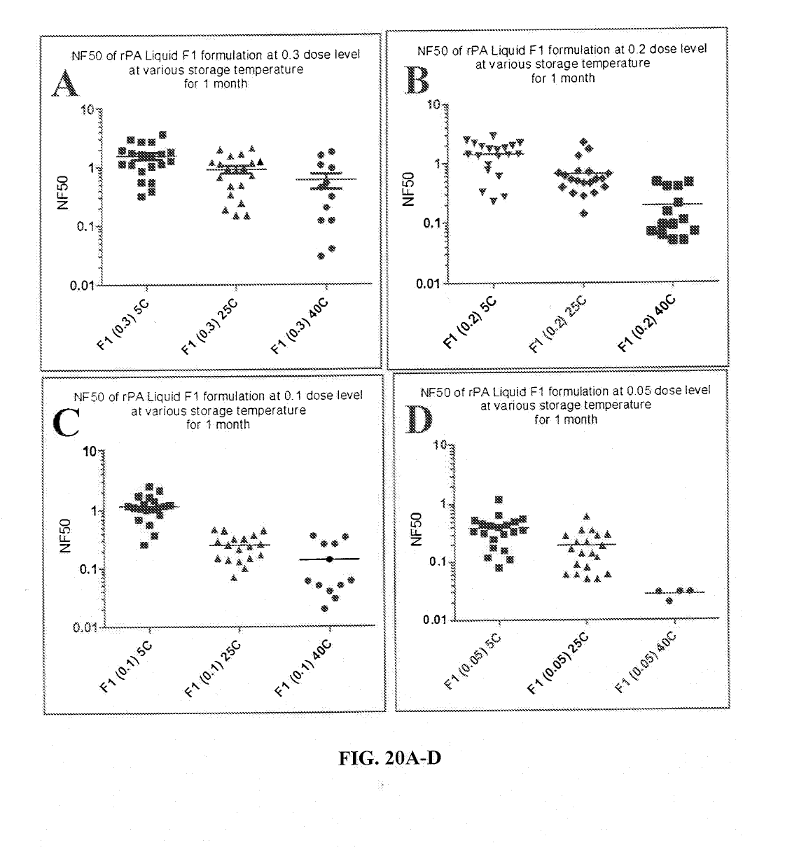

At the 0.3 dose level, the <NF50>gm was found to be 1.26, 0.69 and 0.30 at 5, 25 and 40° C., respectively, and there was a statistically significant difference in all the <NF50>gm at p=0.0023. Similarly, at the lower dose levels of 0.2, 0.1 and 0.05, the <NF50>gm significantly dropped as the storage temperature increases (see FIG. 20A-D). The NF50 was found to decease significantly when the liquid vaccine was stored at 25° C. as compare to 5° C. at all four dose levels, and progressively more at 40° C.

In sum, the immunogenicity data demonstrated the superiority of the lyophilized rPA formulations over the liquid rPA formulation. The lyophilized formulations maintain their immunogenicity at 25 and 40° C. for at least 1 month, while the immunogenicity of the liquid formulation decreases significantly over similar storage conditions.

Like most liquid vaccines, liquid rPA vaccines were found to be unstable at accelerated storage temperatures (e.g., 25 and 40° C.). Liquid rPA vaccine lost its immunogenicity and key physiochemical properties when stored at 40° C. for 1 month. Similarly, the key physiochemical properties of the vaccine were also significantly degraded. The content and purity of vaccine as measured by macrophage lysis assay (MLA), size exclusion chromatography (SEC-HPLC) and anion exchange chromatography (AEX-HPLC) were found to significantly decrease at 25° C. and be undetectable at 40° C. for 1 month. Liquid rPA vaccine was known to be susceptible to deamidation reaction especially when it was adsorbed on aluminum and stored at accelerated temperature.

Three lyophilized formulations were manufactured as lot number: lyoA, lyoB and lyoC. These three new rPA lyophilization formulations had superior stability profile over the rPA liquid vaccines. There was no statistically significant change in purity in all key physiochemical assays (MLA, SEC-HPLC and AEX-HPLC) for all three lots of lyophilized formulations when stored at 4 month for temperatures up to 50° C. In addition, there is no significant drop in immunogenicity (NF50) when the lyophilized vaccines were stored at 1 month for up to 40° C. at four dose levels.

The results herein show that the lyophilized rPA formulations had superior physiochemical and immunological stability profiles over the liquid rPA formulation. The lyophilized formulations maintained all the key physiochemical properties tested by MLA, SEC and AEX at storage temperatures up to 50° C. and over the 4 month storage time period. The lyophilized formulations also maintained immunogenicity at storage temperatures up to 40° C. for at least 1 month. On the contrary, the liquid rPA formulation showed a complete loss of the physiochemical properties (MLA, SEC, and AEX) and a significant drop in immunogenicity after storage for 1 month at 40° C.

### Example 7

### Formulations and Lyophilization with Other Adjuvants

CPG 7909 Bulk Drug Substance (BDS) is packaged as a lyophilized powder in high density polyethylene (HDPE) bottles, heat sealed in multi-layer (mylar, foil) pouches, and stored at −20° C.±5° C.

Glucopyranosyl Lipid Adjuvant (GLA) was obtained from Avanti Polar Lipids, Inc. It was packaged in 2 mL amber glass vials containing 25 mg of lyophilized GLA powder and stored at −20° C.±5° C. GLA is described in Arias et al. (2012) PLoS ONE 7(7):e41144.

Bulk Drug Substance (BDS): 2.81 mg/mL rPA, 0.9% NaCl and buffered in 20 mM Tris-HCl at pH 7.4 was used. It was stored at −80° C. and thawed at 5° C. overnight prior to use.

PolyI PolyC (PIPC) was obtained from InviviGen in 20 mL glass vials as a lyophilized cake containing 50 mg of PIPC. It was stored at 5° C.

TABLE 14
Chemicals and Source

| Chemical Name | Source |
|---|---|
| Tris Hydrochloride, Ultrapure | Amresco |
| 2% ALHYDROGEL (10 mg/mL aluminum) | Brenntag |
| α,α-Trehalose dihydrate | Hayashibara Biochemicals |
| Sodium phosphate, monobasic, Anhydrous | Sigma Aldrich |
| Sodium phosphate, dibasic, hepta-hydrate | BDH |
| Polysorbate 80, N.F. | J. T. Baker |
| L-Alanine | EMD |

TABLE 15
Equipment/Materials

| Name | Source |
|---|---|
| VirTis AdVantage Plus Lyophilizer | SP Scientific |
| Wheaton Serum Vials, Borosilicated Glass | VWR |
| Slotted Rubber Stoppers for Lyo Vials | VWR |
| Flip-Off Crimp Seals | VWR |

Stock Solution Preparations

Two 60% (w/v) solutions of trehalose were prepared in 20 mM Tris and 5 mM NaPi buffers, separately, and were sterile filtered. A 10% (v/v) solution of Polysorbate80 was prepared in DI water and then sterile filtered. Two 12% (w/v) solutions of alanine were prepared in 20 mM Tris and 5 mM NaPi buffers, separately, and were sterile filtered.

One aluminum hydroxide stock solution was buffered by adding 7 mL of IM Tris buffer, pH 7.4, to 343 mL of 2% AlOH (or 10 mg/mL aluminum) and was titrated to pH 7.4. A second aluminum hydroxide stock solution was buffered by adding 1.75 mL of 1 M NaPi buffer, pH 7.0, to 348.25 mL of 2% AlOH and was titrated to pH 7.0. The dilution effect from the buffer addition and subsequent titration was not accounted for in either preparation.

Adjuvant Preparations

GLA adjuvant was prepared in 20 mM Tris-HCl buffer, pH 7.4 by adding 31.2 mg of powder into a 50 mL conical tube and adding 15.6 mL of buffer. The mixture was sonicated for a total of 60 seconds in 10 second intervals with 10 second rests in between to a maximum power of 15 W. A turbid mixture was obtained of 2 mg/mL GLA.

CPG 7909 stocks were prepared in 20 mM Tris, pH 7.4 or 5 mM NaPi, pH 7.0 buffers. In each preparation, about 200 mg of CPG 7909 powder were fully dissolved in a final volume of 10 mL. A clear solution was obtained for both preparations.

A 2 mg/mL stock solution of PIPC was prepared by dissolving a 50 mg lyophilized cake of PIPC in 25 mL of 5 mM NaPi buffer, pH 7.0. The mixture was sonicated for a total of 60 seconds in 10 second intervals with 10 second rests in between to a maximum power of 15 W. A clear solution with no visible particles was obtained.

Formulation Procedure for Blending

TABLE 16
Formulation Blends for Liquid and Lyophilization

| | | Chemical Composition | | | | | | | | | | |
|---|---|---|---|---|---|---|---|---|---|---|---|---|
| | | | | | | | | Tris- | | | | |
| | | rPA | Alum | | Tween | | | HCl | NaPi | | | NaCl |
| Sample | Study | (mg/ | (mg/ | Trehalose | 80 | Adjuvant | Alanine | Buffer | Buffer | | Adjuvant | (residual) |
| nos. | Group | mL) | mL) | (%) | (%) | (mg/mL) | (%) | (mM) | (mM) | pH | Name | (mM) |
| 7-13 | CPG-Liq | 0.15 | 1.5 | 0 | 0.03 | 0.50 | 0 | 20 | 0 | 7.4 | CPG | 8 |
| 14-20 | CPG-Lyo | 0.15 | 1.5 | 20 | 0.03 | 0.50 | 2 | 20 | 0 | 7.4 | CPG | 8 |
| 34-39 | GLA-Liq | 0.15 | 1.5 | 0 | 0.03 | 0.50 | 0 | 20 | 0 | 7.4 | GLA | 8 |
| 40-45 | GLA-Lyo | 0.15 | 1.5 | 20 | 0.03 | 0.50 | 2 | 20 | 0 | 7.4 | GLA | 8 |
| 73-75 | PIPC/CPG-Liq | 0.15 | 1.5 | 0 | 0.03 | 0.50 | 0 | 1.1 | 5 | 7.0 | PIPC + CPG | 8 |
| 76-81 | PIPC/CPG-LYO | 0.15 | 1.5 | 20 | 0.03 | 0.50 | 2 | 1.1 | 5 | 7.0 | PIPC + CPG | 8 |

Table 16 shows the chemical composition of the prepared formulations:

The formulation blends in Table 16 were prepared using buffered stock solutions as shown in Table 17.

TABLE 17
Stock/Excipient Volumes Used for Formulation Blend Preparations

| | Formulation Volumes Added (mL) Stock Solution | | | | | | | | | | |
|---|---|---|---|---|---|---|---|---|---|---|---|
| | rPA | AlOH | | Tween | | | | | | | |
| | Stock | Stock | Trehalose | 80 | Alanine | CpG | GLA | Poly(IC) | NaPi | Tris | |
| | (mg/ | (mg/ | Stock | Stock | Stock | Stock | Stock | Stock | Buffer | Buffer | Final |
| | mL) | mL) | (%) | (%) | (%) | (mg/mL) | (mg/mL) | (mg/mL) | (mM) | (mM) | Volume |
| Study Group | 2.8 | 10 | 60 | 10 | 12 | 20 | 2 | 2 | 5 | 20 | (mL) |
| CPG-Liq | 0.86 | 2.40 | 0 | 0.040 | 0 | 0.40 | 0 | 0 | 0 | 12.30 | 16 |
| CPG-Lyo | 1.07 | 3.00 | 6.67 | 0.050 | 3.333 | 0.50 | 0 | 0 | 0 | 5.38 | 20 |
| GLA-Liq | 0.86 | 2.40 | 0 | 0.040 | 0 | 0 | 4.0 | 0 | 0 | 8.70 | 16 |
| GLA-Lyo | 1.07 | 3.00 | 6.67 | 0.050 | 3.333 | — | 5.0 | 0 | 0 | 0.88 | 20 |
| PIPC/CPG-Liq | 0.86 | 2.40 | 0 | 0.040 | 0 | 0.40 | 0 | 4.0 | 8.30 | 0 | 16 |
| PIPC/CPG-LYO | 1.07 | 3.00 | 0.67 | 0.050 | 3.333 | 0.50 | 0 | 5.0 | 0.38 | 0 | 20 |

The order of addition used for blending all excipients was as follows: aluminum hydroxide→trehalose→TWEEN 80→alanine→buffer→rPA→CPG or GLA adjuvant

A final volume of 20 mL was prepared for samples 14-20, 40-45 and 76-81 for lyophilization. Blends were split into 10 mL glass vials in 2 mL aliquots and set aside for lyophilization.

Lyophilization Procedure

Samples were lyophilized using a VirTis Plus Freeze Dryer. A 73 hour cycle was employed and entered as described in Table 18.

TABLE 18
Lyophilization Cycle

| | | Temp | Time | Ramp | Vacuum |
|---|---|---|---|---|---|
| # | Step | (° C.) | (Hr) | Hold | (mTorr) |
| 1 | Thermal | −60 | fast | — | — |
| 2 | Treatment | −60 | 2 | H | |
| 3 | | −28 | 1 | R | |
| 4 | | −28 | 20 | H | |
| 5 | Extra | −28 | 10 | H | 20 |
| | Freezing | | | | |
| 6 | Primary | 25 | 8 | R | 20 |
| 7 | Drying | 25 | 10 | H | |
| 8 | | 30 | 1 | R | |
| 9 | | 30 | 5 | H | |
| 10 | Secondary | 35 | 1 | R | |
| 11 | Drying | 35 | 5 | H | |
| 12 | | 40 | 1 | R | |
| 13 | | 40 | 4 | H | |
| 14 | | 45 | 1 | R | |
| 15 | | 45 | 4 | H | |

| | | | | |
|---|---|---|---|---|
| Post-Drying | 25 | — | — | 3000 |

Summary of Data

The immunological response of the six vaccines formulations was tested in mice (n=10) with one immunization using IP route. The serum was collected 28 days after immunization. TNA data at dose level=0.4 was determined as described in Example 3 and the results are summarized in Table 19.

The mean NF50 was found to be 53.6, 48.9, 69.9, 64.7, 89.4 and 77.3 for CPG-liq, CPG-lyo, GLA-liq, GLA-lyo, PIPC/CPG-liq and PIPC/CPG-lyo samples, respectively. There is no statistical different in the mean NF50 of the liquid versus the lyophilized formulations for CPG, GLA and PIPC/CPG. The data demonstrated the lyophilized formulation and process is capable of maintaining the immunogenicity, even in the presence and of other adjuvants.

TABLE 19
NF50 of liquid versus lyophilization formulations of CPG,
GLA and PIPC/CPG adjuvant containing vaccines

| | CPG- | CPG- | GLA- | GLA- | PIPC/ | PIPC/ |
|---|---|---|---|---|---|---|
| n | Liq | Lyo | Liq | Lyo | CPG-Liq | CPG-Liq |
| | NF50 (DL = 0.4) | | | | | |
| 1 | 51.2 | 43.1 | 114.4 | 108.9 | 91.6 | 62.8 |
| 2 | 106.3 | 29.2 | 115.2 | 23.7 | 100.8 | 67.2 |
| 3 | 103.9 | 77.4 | 43.1 | 65.1 | 114.5 | 119.2 |
| 4 | 33.1 | 17.2 | 50.3 | 60.9 | 74.6 | 84.0 |
| 5 | 10.3 | 47.8 | 122.0 | 83.3 | 19.4 | 63.2 |
| 6 | 37.3 | 31.4 | 55.9 | 29.2 | 249.5 | 68.6 |
| 7 | 47.6 | 51.8 | 50.1 | 115.8 | 61.0 | 84.8 |
| 8 | 54.7 | 66.4 | 33.3 | 79.3 | 65.3 | 62.4 |
| 9 | 42.8 | 36.5 | 81.7 | 67.9 | 29.8 | 79.4 |
| 10 | 48.9 | 87.9 | 32.5 | 12.9 | 87.4 | 81.2 |
| Mean | 53.6 | 48.9 | 69.9 | 64.7 | 89.4 | 77.3 |
| Stdev | 29.9 | 22.5 | 35.5 | 34.6 | 63.6 | 17.3 |
| P value T-test | 0.695 | | 0.746 | | 0.573 | |
| | Log NF50 (DL = 0.4) | | | | | |
| 1 | 1.7 | 1.6 | 2.1 | 2.0 | 2.0 | 1.8 |
| 2 | 2.0 | 1.5 | 2.1 | 1.4 | 2.0 | 1.8 |
| 3 | 2.0 | 1.9 | 1.6 | 1.8 | 2.1 | 2.1 |
| 4 | 1.5 | 1.2 | 1.7 | 1.8 | 1.9 | 1.9 |
| 5 | 1.0 | 1.7 | 2.1 | 1.9 | 1.3 | 1.8 |
| 6 | 1.6 | 1.5 | 1.7 | 1.5 | 2.4 | 1.8 |
| 7 | 1.7 | 1.7 | 1.7 | 2.1 | 1.8 | 1.9 |
| 8 | 1.7 | 1.8 | 1.5 | 1.9 | 1.8 | 1.8 |
| 9 | 1.6 | 1.6 | 1.9 | 1.8 | 1.5 | 1.9 |
| 10 | 1.7 | 1.9 | 1.5 | 1.1 | 1.9 | 1.9 |
| Mean Log | 1.7 | 1.6 | 1.8 | 1.7 | 1.9 | 1.9 |
| GeoMean | 45.6 | 44.1 | 62.2 | 53.7 | 72.4 | 75.8 |
| Stdev | 0.3 | 0.2 | 0.2 | 0.3 | 0.3 | 0.1 |

| | | | |
|---|---|---|---|
| P value T-test | 0.898 | 0.607 | 0.849 |

### Example 8

### Immunogenicity of Lyophilized rPA Vaccines

This Examples compares liquid vaccine freshly made vs lyophilized vaccine stored at 5 and 50° C. for 1 month and compares CPG formulations made in NaPi (pH 7.0) vs Citric (pH 5.5) buffers.

There are four formulation evaluated under this study:

rPA alum in NaPi (pH 7.0)

rPA alum+CPG in NaPi (pH 7.0)

rPA alum+CPG in Critic (pH 5.5)

rPA alum+GLA in Tris (pH 7.4)

### Stock Solution Preparations

Three 60% (w/v) solutions of trehalose were prepared, one in 20 mM Tris (pH 7.4), a second in 5 mM NaPi buffers (pH 7.0), and a third in 20 mM Na-Citrate (pH 5.5). A 10% (v/v) solution of Polysorbate80 was prepared by mixing 10 mL of concentrated Tween80 in 90 mL of DI water. Three 12% (w/v) solutions of alanine were prepared, one in 20 mM Tris (pH 7.4), a second in 5 mM NaPi buffers (pH 7.0), and a third in 20 mM Na-Citrate (pH 5.5) and all were sterile filtered.

Three aluminum hydroxide stock solutions were buffered by adding IM buffers into the 2% AlOH. The resulting stocks were titrated to the desired pH to match the buffer in use. The dilution effect from the buffer addition and subsequent titration was not accounted for in any of the preparations.

TABLE 19
Formulations Prior to Lyophilization

| | | Storage | rPA | Aluminum | Trehal. | Alanine | CPG | GLA | TWEEN |
|---|---|---|---|---|---|---|---|---|---|
| # | Buffer | Condition | mg/mL | mg/mL | % | % | mg/mL | mg/mL | 80 (%) |
| 1 | 5 mM | Liquid Fresh | 0.5 | 5.0 | 20.0 | 2.0 | — | — | 0.025 |
| 2 | NaPI | Lyo 5° C., 1 mo. | | | | | | | |
| 3 | pH 7.0 | Lyo 50° C., 1 mo | | | | | | | |
| 4 | 5 mM | Liquid Fresh | 0.45 | 4.5 | 20.0 | 2.0 | 1.5 | — | 0.025 |
| 5 | NaPI | Lyo 5° C., 1 mo. | | | | | | | |
| 6 | pH 7.0 | Lyo 50° C., 1 mo | | | | | | | |
| 7 | Na-Citrate | Liquid Fresh | 0.45 | 4.5 | 20.0 | 2.0 | 1.5 | — | 0.025 |
| 8 | pH 5.5 | Lyo 5° C., 1 mo. | | | | | | | |
| 9 | | Lyo 50° C., 1 mo | | | | | | | |
| 10 | 20 mM | Liquid Fresh | 0.45 | 4.5 | 20.0 | 0.0 | — | 0.30 | 0.025 |
| 11 | Tris-HCL | Lyo 5° C., 1 mo. | | | | | | | |
| 12 | pH 7.4 | Lyo 50° C., 1 mo | | | | | | | |

Each formulation was blended and then lyophilized at 2 mL per vial, using the lyophilization process as described in Example 7.

TABLE 20
Formulations Prior to Lyophilization

| | | Storage | rPA | Aluminum | Trehal. | Alanine | CPG | GLA | TWEEN |
|---|---|---|---|---|---|---|---|---|---|
| # | Buffer | Condition | mg/mL | mg/mL | % | % | mg/mL | mg/mL | 80 (%) |
| 1 | 5 mM | Liquid Fresh | 0.15 | 1.5 | 6.06 | 0.61 | 0 | 0 | .008 |
| 2 | NaPI | Lyo 5° C., 1 mo. | | | | | | | |
| 3 | pH 7.0 | Lyo 50° C., 1 mo | | | | | | | |
| 4 | 5 mM | Liquid Fresh | 0.15 | 1.5 | 6.67 | 0.61 | .5 | 0 | .008 |
| 5 | NaPI | Lyo 5° C., 1 mo. | | | | | | | |
| 6 | pH 7.0 | Lyo 50° C., 1 mo | | | | | | | |
| 7 | Na-Citrate | Liquid Fresh | 0.15 | 1.5 | 6.67 | 0.67 | .5 | 0 | .008 |
| 8 | pH 5.5 | Lyo 5° C., 1 mo. | | | | | | | |
| 9 | | Lyo 50° C., 1 mo | | | | | | | |
| 10 | 20 mM | Liquid Fresh | 0.15 | 1.5 | 6.67 | 0 | 0 | 0.1 | .008 |
| 11 | Tris-HCL | Lyo 5° C., 1 mo. | | | | | | | |
| 12 | pH 7.4 | Lyo 50° C., 1 mo | | | | | | | |

### Animal Model

Each animal receive 0.5 mL of 1/16 dilution of the listed formulations. 5 Female/5 Male Guinea pig per group (n=10). IM immunization was performed on Day 0 and Day 14. Blood collection was performed on day 14, 28 and 35. The TNA data at day 28 was analyzed and presented.

### NF50 Data

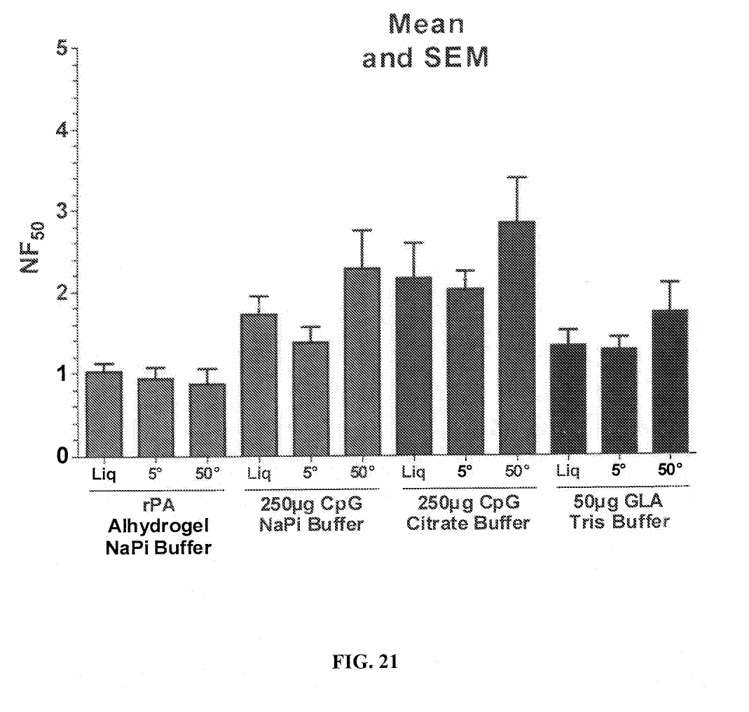

FIG. 21 shows the NF50 and the standard deviation of mean of the 12 formulations.

Table 21 shows the numerical values of each mouse of the 12 formulations.

TABLE 21

| | NF50 and LogNF50 For Each Mouse For Each of the 12 Formulations. | | | | | | | | | | | |
|---|---|---|---|---|---|---|---|---|---|---|---|---|
| | | | | 250 μg | 250 μg | 250 μg | 250 μg | 250 μg | 250 μg | 50 μg | 50 μg | 50 μg |
| | rPA | rPA | rPA | CpG | CpG | CpG | CpG | CpG | CpG | GLA | GLA | GLA |
| | Alhydrogel | Alhydrogel | Alhydrogel | NaPi | NaPi | NaPi | Citrate | Citrate | Citrate | Tris | Tris | Tris |
| n | NaPi Liq | NaPi 5° C. | NaPi 50° C. | Liq | 5° C. | 50° C. | Liq | 5° C. | 50° C. | Liq | 5° C. | 50° C. |
| | NF50 (DL = 0.063) | | | | | | | | | | | |
| 1 | 1.4 | 0.9 | 1.0 | 2.9 | 0.9 | 4.8 | 1.5 | 1.9 | 2.3 | 1.1 | 2.2 | 2.4 |
| 2 | 1.1 | 0.7 | 0.4 | 2.5 | 2.1 | 1.9 | 4.6 | 2.4 | 3.3 | 2.3 | 0.8 | 2.4 |
| 3 | 1.0 | 1.8 | 1.5 | 1.2 | 0.8 | 5.1 | 3.0 | 2.8 | 4.3 | 2.1 | 1.2 | 1.8 |
| 4 | 0.6 | 1.0 | 1.0 | 1.8 | 1.9 | 2.6 | 4.2 | 2.9 | 4.2 | 1.7 | 2.0 | 4.2 |
| 5 | 1.1 | 1.3 | 2.0 | 2.4 | 2.6 | 1.8 | 0.9 | 1.6 | 2.8 | 1.4 | 0.8 | 2.1 |
| 6 | 1.3 | 1.4 | 0.2 | 1.7 | 1.0 | 1.5 | 1.5 | 1.3 | 1.9 | 1.4 | 1.2 | 0.5 |
| 7 | 1.3 | 1.0 | 0.8 | 1.9 | 1.4 | 1.4 | 0.8 | 2.7 | 6.3 | 0.9 | 0.9 | 1.6 |
| 8 | 0.7 | 0.4 | 0.2 | 0.9 | 1.4 | 1.1 | 1.5 | 0.9 | 1.2 | 1.3 | 1.1 | 0.6 |
| 9 | 0.4 | 0.5 | 1.0 | 0.8 | 0.6 | 1.9 | 1.5 | 2.3 | 1.5 | 0.6 | 1.1 | 1.1 |
| 10 | 1.3 | 0.5 | 0.5 | 1.2 | 0.8 | 0.6 | 2.0 | 1.2 | 0.6 | 0.4 | 1.3 | 0.4 |
| Mean | 1.0 | 0.9 | 0.9 | 1.7 | 1.4 | 2.3 | 2.2 | 2.0 | 2.8 | 1.3 | 1.3 | 1.7 |
| SD | 0.3 | 0.5 | 0.6 | 0.7 | 0.7 | 1.5 | 1.4 | 0.7 | 1.7 | 0.6 | 0.5 | 1.2 |
| % CV | 33.9 | 49.1 | 66.6 | 40.8 | 48.1 | 65.8 | 62.6 | 36.0 | 61.1 | 46.4 | 38.6 | 67.5 |
| P Value | 0.770 | | | 0.152 | | | 0.356 | | | 0.412 | | |
| (ANOVA) | | | | 0.132 | | | | | | | | |
| | Log NF50 (DL = 0.063) | | | | | | | | | | | |
| 1 | 0.15 | −0.03 | 0.00 | 0.46 | −0.03 | 0.68 | 0.19 | 0.28 | 0.36 | 0.03 | 0.35 | 0.38 |
| 2 | 0.04 | −0.19 | −0.44 | 0.39 | 0.31 | 0.29 | 0.67 | 0.38 | 0.52 | 0.36 | −0.12 | 0.38 |
| 3 | −0.02 | 0.26 | 0.17 | 0.09 | −0.08 | 0.71 | 0.47 | 0.45 | 0.63 | 0.32 | 0.09 | 0.26 |
| 4 | −0.20 | −0.01 | 0.00 | 0.25 | 0.29 | 0.41 | 0.63 | 0.47 | 0.63 | 0.24 | 0.30 | 0.62 |
| 5 | 0.06 | 0.10 | 0.31 | 0.38 | 0.42 | 0.26 | −0.07 | 0.22 | 0.45 | 0.13 | −0.12 | 0.33 |
| 6 | 0.11 | 0.14 | −0.74 | 0.24 | 0.01 | 0.19 | 0.19 | 0.12 | 0.27 | 0.16 | 0.09 | −0.35 |
| 7 | 0.12 | −0.02 | −0.09 | 0.27 | 0.15 | 0.16 | −0.12 | 0.43 | 0.80 | −0.06 | −0.03 | 0.21 |
| 8 | −0.13 | −0.41 | −0.61 | −0.05 | 0.13 | 0.04 | 0.19 | −0.07 | 0.06 | 0.12 | 0.06 | −0.22 |
| 9 | −0.45 | −0.34 | 0.01 | −0.11 | −0.21 | 0.27 | 0.19 | 0.37 | 0.19 | −0.22 | 0.04 | 0.04 |
| 10 | 0.11 | −0.30 | −0.27 | 0.08 | −0.11 | −0.24 | 0.29 | 0.09 | −0.23 | −0.40 | 0.10 | −0.36 |
| Mean Log | −0.02 | −0.08 | −0.17 | 0.20 | 0.09 | 0.28 | 0.26 | 0.27 | 0.37 | 0.07 | 0.08 | 0.13 |
| Geo Mean | 0.95 | 0.83 | 0.68 | 1.58 | 1.23 | 1.89 | 1.83 | 1.88 | 2.33 | 1.17 | 1.19 | 1.34 |
| SD | 0.19 | 0.22 | 0.34 | 0.19 | 0.21 | 0.28 | 0.26 | 0.18 | 0.31 | 0.24 | 0.15 | 0.3 |

| | | | | |
|---|---|---|---|---|
| P Value | 0.472 | 0.201 | 0.603 | 0.847 |

| | |
|---|---|
| (ANOVA) | 0.210 |

The NF50 data shows superior stability of the four lyophilized formulations. It also demonstrated the robustness of the formulations.

No statistical difference of NF50 mean and Geomean among liquid fresh, lyo (5 and 50° C. for 1 month) for all four formulations: (see Table 21)

No statistical difference of mean and geomean of NF50 between NaPi vs Citric buffer for rPA alum+CPG formulation. (see Table 21)

### Example 9

### Formulations with Influenza Antigen(s)

Formulations containing influenza antigen(s) are formulated using methods similar to the disclosed herein for rPA formulations, except for the presence of influenza antigen(s) and the absence of rPA antigens.

Examples of formulations containing influenza antigens are listed in Table 20.

TABLE 22
Examples of Formulations Containing Influenza
Antigen for Lyophilization

| Formu- | Influenza | Alum | | | | Liq. |
|---|---|---|---|---|---|---|
| lation | Antigen | mg/ | | Amino | TWEEN | Lyoph. |
| # | mg/ml | ml | Sugar | Acid | 80 | Vol. |
| 1 | 0.15 | 1.5 | 20% Trehalose | none | 0.025% | 2 mL |
| 2 | 0.15 | 1.5 | 30% Trehalose | none | 0.025% | 2 mL |
| 3 | 0.15 | 1.5 | 20% Trehalose | 2% Ala | 0.025% | 2 mL |
| 4 | 0.15 | 1.5 | 30% Trehalose | 2% Ala | 0.025% | 2 mL |
| 5 | 0.15 | 1.5 | 20% Trehalose | 2% Gly | 0.025% | 2 mL |
| 6 | 0.15 | 1.5 | 30% Trehalose | 2% Gly | 0.025% | 2 mL |
| 7 | 0.15 | 1.5 | 20% Trehalose | 2% Arg | 0.025% | 2 mL |
| 8 | 0.15 | 1.5 | 30% Trehalose | 2% Arg | 0.025% | 2 mL |
| 9 | 0.15 | 1.5 | 10% Trehalose | 2% Ala | 0.025% | 2 mL |
| 10 | 0.15 | 1.5 | 10% Trehalose | 2% Gly | 0.025% | 2 mL |
| 11 | 0.15 | 1.5 | 10% Trehalose | 2% Arg | 0.025% | 2 mL |
| 12 | 0.5 | 5 | 20% Trehalose | none | 0.025% | 2 mL |
| 13 | 0.5 | 5 | 30% Trehalose | none | 0.025% | 2 mL |
| 14 | 0.5 | 5 | 20% Trehalose | 2% Ala | 0.025% | 2 mL |
| 15 | 0.5 | 5 | 30% Trehalose | 2% Ala | 0.025% | 2 mL |
| 16 | 0.5 | 5 | 20% Trehalose | 2% Gly | 0.025% | 2 mL |
| 17 | 0.5 | 5 | 30% Trehalose | 2% Gly | 0.025% | 2 mL |
| 18 | 0.5 | 5 | 20% Trehalose | 2% Arg | 0.025% | 2 mL |
| 19 | 0.5 | 5 | 30% Trehalose | 2% Arg | 0.025% | 2 mL |
| 20 | 0.5 | 5 | 10% Trehalose | 2% Ala | 0.025% | 2 mL |
| 21 | 0.5 | 5 | 10% Trehalose | 2% Gly | 0.025% | 2 mL |
| 22 | 0.5 | 5 | 10% Trehalose | 2% Arg | 0.025% | 2 mL |

Two sets of formulations are made, one in 5 mM NaPi, pH 7.0 buffer or 20 mM Tris, pH 7.4 buffer. The influenza antigen is an influenza hemagglutinin. Formulations may also contain another adjuvant such as CPG at 0.5 mg/mL, PIPC at 0.5 mg/mL, GLA at 0.1 mg/Ml or a combination thereof.

Each formulation is lyophilized, 2 mL per vial, using the lyophilization process as described in Example 7.

Each formulation is tested in immunogenicity studies, stability studies and/or efficacy studies.

What is claimed is:

1. A composition for preparation of a lyophilized vaccine comprising at least one antigen adsorbed to an aluminum adjuvant and at least 20% (w/v) non-reducing sugar.

2. A temperature stable liquid vaccine composition comprising at least one antigen adsorbed to an aluminum adjuvant and at least 20% (w/v) sugar.

3. A composition comprising, prior to lyophilization, at least one antigen absorbed to an aluminum adjuvant and at least 20% (w/v) non-reducing sugar, wherein after reconstitution the non-reducing sugar is at least 6% (w/v).

4. The composition of any one of claims 1-3, wherein the composition comprises a surfactant.

5. A composition comprising at least one antigen adsorbed to an aluminum adjuvant, a surfactant and at least 15% (w/v) sugar for preparation of a lyophilized vaccine.

6. A temperature stable liquid vaccine composition comprising at least one antigen adsorbed to an aluminum adjuvant, a surfactant and at least 15% (w/v) sugar.

7. The composition of any one of claims 1-6, wherein said composition further comprises an amino acid.

8. A composition for preparation of a lyophilized vaccine comprising at least one antigen adsorbed to an aluminum adjuvant, a surfactant, an amino acid and at least 10% (w/v) sugar.

9. A stable liquid vaccine composition comprising at least one antigen adsorbed to an aluminum adjuvant, a surfactant, an amino acid and at least 10% (w/v) sugar.

10. The composition of any one of claims 1-9, wherein the aluminum adjuvant is selected from the group consisting of aluminum phosphate and aluminum sulfate.

11. The composition of any one of claims 1-9, wherein the aluminum adjuvant is aluminum hydroxide.

12. The composition of claim 11, wherein the composition contains about 0.5 to 1.5 mg/ml aluminum hydroxide.

13. The composition of claim 12, wherein the composition contains about 0.5 mg/ml aluminum hydroxide.

14. The composition of claim 12, wherein the composition contains about 1.5 mg/ml aluminum hydroxide.

15. The composition of any one of claims 4-14, wherein the surfactant is selected from the group consisting of polysorbate 80 and polysorbate 20.

16. The composition of claim 15, wherein the surfactant is polysorbate 80.

17. The composition of any one of claims 4-16, wherein the composition contains about 0.020% or 0.025% (w/v) surfactant.

18. The composition of claims 1-17, wherein the sugar is a non-reducing sugar selected from the group consisting of trehalose, sucrose, and a combination thereof.

19. The composition of claim 18, wherein the sugar is trehalose.

20. The composition of claim 18, wherein the sugar is sucrose.

21. The composition of claim 18, wherein the sugar is trehalose and sucrose.

22. The composition of any one of claims 5-21, wherein the composition contains about 15-40% (w/v) sugar.

23. The composition of any one of claim 1-22, wherein the composition contains about 20-40% (w/v) sugar.

24. The composition of claim 23, wherein the composition contains about 20-35% (w/v) sugar.

25. The composition of claim 24, wherein the composition contains about 25-40% (w/v) sugar.

26. The composition of any one of claims 1-25, wherein the composition contains greater than about 20%, 21%, 22%, 23%, 24% and 25% (w/v) sugar.

27. The composition of any one of claims 1-26, wherein the antigen is an Anthrax antigen.

28. The composition of claim 27, wherein the Anthrax antigen is protective antigen.

29. The composition of claim 28, wherein the protective antigen is at least about 80% identity to the polypeptide of SEQ ID NO: 2.

30. The composition of any one of claims 28-29, wherein the composition comprises about 150-500 μg/ml protective antigen.

31. The composition of claim 30, wherein the composition comprises about 150, 175, 200, 225, 250, 275, 300, 325, 400, 375, 400, 425, 450, 475 or 500 μg/ml protective antigen

32. The composition of claim 31, wherein the Anthrax antigen is a cell-free filtrate from an avirulent B. anthracis strain.

33. The composition of claim 32, wherein the avirulent B. anthracis strain is V770-NP1-R.

34. The composition of any one of claims 7-33 wherein said amino acid is selected from the group consisting of arginine, alanine, proline, glycine, and any combination thereof.

35. The composition of claim 34, wherein the composition contains about 0.5-4% (w/v) alanine or arginine.

36. The composition of claim 40, wherein the composition contains about 2% (w/v) alanine or arginine.

37. The composition of claim 34, wherein the composition contains about 6-12% (w/v) glycine.

38. The composition of claim 37, wherein the composition contains about 10% (w/v) glycine.

39. The composition of any one of claims 1, 3-5, 7, 8, and 10-38, wherein said composition is subjected to sublimation under vacuum to produce a lyophilized composition.

40. A lyophilized composition, which is lyophilized from the composition of any one of claims 1, 3-5, 7, 8, and 10-38.

41. A reconstituted composition comprising the lyophilized composition of claim 39 or 40 reconstituted in an aqueous solution.

42. The reconstituted composition of claim 41, wherein the aqueous solution is selected from the group consisting water, Tris EDTA (TE), phosphate buffered saline (PBS), Tris buffer or saline.

43. The composition of any one of claims 1, 3-5, 7, 8, and 10-38, wherein the composition retains at least 80%, at least 90% or at least 95% purity after storage in lyophilized form for at least 4 months at 50° C.

44. The composition of any one of claims 1, 3-5, 7, 8, and 10-38, wherein the composition retains at least 80%, at least 90% or at least 95% immunogenicity after storage in lyophilized form for at least 1 months at 40° C.

45. A method of vaccinating a subject against a pathogen comprising administering the composition of any one of claims 1-44.

46. A method of vaccinating a subject against a pathogen comprising administering to a subject a pharmaceutical composition reconstituted from the lyophilized composition of claim 39 or 40.

47. A method of producing a potent, alum based frozen vaccine comprising suspending a composition comprising at least about 10% sugar and an antigen adsorbed to an aluminum adjuvant and freezing said composition at a rate sufficient to freeze the suspended composition before sedimentation occurs.

48. The method of claim 47, wherein said composition contains at least about 15% sugar.

49. The method of claim 47, wherein said composition contains at least about 20% sugar.

50. A method of preparing a stable lyophilized composition, comprising lyophilizing a composition of any one of claims 1, 3-5, 7, 8, and 10-38, wherein the stability of the reconstituted lyophilized composition is measured by microphage lysis assay (MLA), size exclusion chromatography (SEC-HPLC) and/or anion exchange chromatography (AEX-HPLC).


SEQUENCE LISTING (3 sequences)

SEQ ID NO: 1 - protein, 735 aa, Artificial Sequence
  Synthetic polypeptide
EVKQENRLLNESESSSQGLLGYYFSDLNFQAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 2 - protein, 706 aa, Artificial Sequence
  Synthetic polypeptide
QAPMVVTSSTTGDLSIPSSELENIPSENQYFQSAIWSGFIKVKKSDEYTFATSADNHVTMWVDDQEVINKASNSNKIRLEKGRLYQIKIQYQRENPTEKGLDFKLYWTDSQNKKEVISSDNLQLPELKQKSSNSRKKRSTSAGPTVPDRDNDGIPDSLEVEGYTVDVKNKRTFLSPWISNIHEKKGLTKYKSSPEKWSTASDPYSDFEKVTGRIDKNVSPEARHPLVAAYPIVHVDMENIILSKNEDQSTQNTDSQTRTISKNTSTSRTHTSEVHGNAEVHASFFDIGGSVSAGFSNSNSSTVAIDHSLSLAGERTWAETMGLNTADTARLNANIRYVNTGTAPIYNVLPTTSLVLGKNQTLATIKAKENQLSQILAPNNYYPSKNLAPIALNAQDDFSSTPITMNYNQFLELEKTKQLRLDTDQVYGNIATYNFENGRVRVDTGSNWSEVLPQIQETTARIIFNGKDLNLVERRIAAVNPSDPLETTKPDMTLKEALKIAFGFNEPNGNLQYQGKDITEFDFNFDQQTSQNIKNQLAELNATNIYTVLDKIKLNAKMNILIRDKRFHYDRNNIAVGADESVVKEAHREVINSSTEGLLLNIDKDIRKILSGYIVEIEDTEGLKEVINDRYDMLNISSLRQDGKTFIDFKKYNDKLPLYISNPNYKVNVYAVTKENTIINPSENGDTSTNGIKKILIFSKKGYEIG

SEQ ID NO: 3 - protein, 4 aa, Artificial Sequence
  Synthetic peptide
RKKR